(19) United States
(12) Patent Application Publication
(10) Pub. No.: US 2016/0034665 A1
(43) Pub. Date: Feb. 4, 2016

(54) SYSTEM AND METHOD FOR PERSONALIZED HEMODYNAMICS MODELING AND MONITORING

(71) Applicant: CARDIOART TECHNOLOGIES LTD., Yehuda (IL)

(72) Inventors: Lev Adirovich, Gan Yavne (IL); Alexander Roytvarf, Rishon le-Zion (IL)

(21) Appl. No.: 14/775,668

(22) PCT Filed: Mar. 13, 2014
(86) PCT No.: PCT/IB2014/000331

§ 371 Date: Sep. 11, 2015

Related U.S. Application Data
(60) Provisional application No. 61/782,597, filed on Mar. 14, 2013.

(51) Int. Cl. G06F 19/00; A61B 6/00; A61B 6/03; A61B 8/08

(52) U.S. Cl. CPC G06F 19/3437; A61B 8/0883; A61B 8/488; A61B 6/503; A61B 6/032; A61B 6/037

(57) ABSTRACT

The present invention relates to a system and a method for evaluating cardiac parameters and forming a personalized cardiac model, and in particular, to such a system and method in which a personalized cardiac model is abstracted and utilized for monitoring cardiac parameters.

### CROSS-REFERENCE TO RELATED APPLICATIONS

This application claims the benefit of priority from U.S. Provisional Patent Application No. 61/782,597 filed on Mar. 14, 2013, titled “SYSTEM AND METHOD FOR PERSONALIZED HEMODYNAMICS MODELING AND MONITORING,” which is incorporated by reference herein in its entirety.

### FIELD OF THE INVENTION

The present invention relates to a system and a method for evaluating cardiac parameters and forming a personalized cardiac model, and in particular, to such a system and method in which a personalized cardiac model is abstracted and utilized for monitoring cardiac parameters.

### BACKGROUND OF THE INVENTION

Scientists have been attempting to predict the function of the cardiovascular system and in particular the heart for many years. These attempts have been varied in methods across various scientific fields, Mathematical modeling has been one approach that has attempted to predict the functionality of the heart both as in its various parts and as a whole system. However modeling the heart is complex as there are a great number of variable that are both, dynamic and correlated. Most cardiac variables are not readily predictable and are further complicate by being greatly dependent on various factors such as human behavior, various non-cardiac diseases, environmental conditions, heart remodeling and non-predictive events.

The cardiovascular and/or circulatory system works as a closed system, therefore an effect of one part of the system in-turn affect all other parts of the system, leading to its complexity and dynamic nature. For example, if a person's blood pressure rises (hypertension) then there is a corresponding pressure decrease in the venous system, the decrease is much smaller than the increase in the arterial side because of the fact that venous vasculature is more compliant than the arterial vasculature. Within the circulatory system the key component is the heart. Any change to any component of the heart will have an effect felt throughout the entire system.

The primary function of a heart is to deliver oxygenated blood to tissue throughout the body. This function is accomplished in several successive steps, each relating to a particular chamber of the heart anatomy. Initially, deoxygenated blood is received in the right auricle of the heart. This deoxygenated blood is pumped by the right ventricle of the heart to the lungs where the blood is oxygenated. The oxygenated blood is initially received in the left auricle of the heart and ultimately pumped by the left ventricle of the heart throughout the body. The left ventricular chamber of the heart is of particular importance in this process as it is responsible for pumping the oxygenated blood through the aortic′ valve and ultimately throughout the entire vascular system.

Modeling of the cardiovascular systems requires that each of the heart's chambers as well as the concerted activity be simultaneously accounted for. In particular proper modeling of the cardiovascular system should explain and/or account for different anomalies of the cardiovascular system, for example hypertension and heart failure.

The most common cardiovascular anomalies reported today remain hypertension and congestive heart failure. These well-known hemodynamic disorders reflect changes and/or anomalies in the balances between the forces and physical mechanisms involved in the circulatory system, and may be indicative of changes associated with the heart's chambers and/or overall anatomy.

In order to solve problems associated with the functionality of the heart and in order to understand the causes leading to them and/or accurately monitoring cardiovascular changes such as hypertension, most researchers have been breaking down the problem into more manageable problems, placing their focus and attention only on a particular aspect of the cardiovascular system and modeling it, for example the left ventricle.

For example, some researchers model the hemodynamics of the large human arteries, other researchers have only modeled a heart geometry and a muscle fiber organization and some researchers have studied the cellular physiology and biochemical processes inside the cardiomyocyte.

For modeling the whole cardiovascular system, the investigators generally use the lumped parameter method, in which the average pressure and flow are modeled by the electric potential and the current, respectively. An arterial vessel is described by using impedance, which is represented by an appropriate combination of resistors, capacitors and inductors.

Despite the pioneering work of W. Harvey, L. Euler, D. Bernoulli, J. Poiseuille and other scientists, comprehensive models that characterize the complete cardiovascular system and enable a computerized numerical solution based on fundamental physical (fluid dynamics and elasticity) laws are not sufficiently developed for usage in medical practice or other real life applications.

Most mathematical models generally simulate a particular aspect of a disease or otherwise healthy biological process, and do not provide the global integrative process at large. For example, mathematical modeling for cardiac output, blood pressure, ejection function and the like cardio-physiological processes are individually known in the art. However, the ability to combine and correlate these seemingly individualistic models into a comprehensive model able to analyze, predict or explain a biological phenomenon at a specific biological level such as organ has been sought after however remains outstanding.

US Patent Publication No. 2011/0144967 to Adirovich, the contents of which is incorporated herein by reference as if fully set herewith, teaches an integrated modeling system that models the entire heart however it does not provide a method capable of producing a stabilized and personalized hemodynamic monitoring capable Of identifying hemodynamic parameters that are not readily measurable.

### SUMMARY OF THE INVENTION

The present invention overcomes the deficiencies of the background by providing a system and method for evaluating hemodynamic and/or cardiac parameters and forming a personalized cardiac model, that is then utilized for monitoring cardiac parameters. The cardiac modeling of the present invention is characterized in that the model is abstracted around events of the cardiac cycle wherein each event of the cardiac cycle is individually modeled to form a personal hemodynamic model of the entire heart. Most preferably an individual cardiac cycle is divided into a set of 15 cases and/or events. Most preferably each of the 15 cardiac cycle events is modeled with a plurality of cardiac functions.

An embodiment of the present invention provides a method for monitoring a plurality of cardiac parameters in a two phase process. The two phase process comprising a first phase wherein a personalized hemodynamic model is abstracted relative to a primary data set comprising a plurality of cardiac parameters; and a second phase where the personalized cardiac model is used to monitor a plurality of monitored cardiac parameters. Optionally and most preferably the monitored cardiac parameters provide insight into hemodynamic and/or cardiac parameters that are dynamically changing during the cardiac cycle that are not readily available and/or attainable by non-invasive means. Most preferably the output monitored cardiac parameters are based on a monitoring input set comprising at least one input monitoring cardiac parameters to infer a plurality of monitored hemodynamic parameters. Optionally the monitoring input parameters may for example include but is not limited to any dynamic cardiac parameters pressure, diameter of vessels, velocity inside chamber, ventricular volume, velocity in the vessel, velocity through valves, changing parameter during cycle, the like, or any combination thereof. Optionally the monitoring input parameter may for example be obtained from a direct measured parameter, an inferred parameter, from a graph or the like.

Optionally a plurality of input monitoring cardiac parameter may be utilized.

Within the context of this application the term auxiliary device refers to any device that may communicate (receive or send) and/or exchange data with the system of the present invention. Auxiliary device may for example include but is not limited to an image processing device, computer, server, a mobile communication device, a smartphone, an implanted device, a health care-giver system, health care-giver database, decision support system, echocardiograph, ultrasound, CT, MRI, PET, image processor, non-imagery measuring device, sensor, implanted sensor, data storage device, online monitoring device, sphygmomanometer, blood pressure device, direct catheterization device, electronic devices, implanted device, electrocardiograph (‘ECG’ or ‘EKG’), laboratory testing device, blood works parameters

Within the context of this application the term cardiac functions refers to any function and/or mathematical model that reiterates at least one aspect of cardiovascular physiology.

Within the context of this application the term Primary Set refers to the set that is used to abstract the model comprises: input measured set, complementary randomized data set, model set portion

Within the context of this application the term input measured set refers to a set of measured parameters most preferably from imagery data, echocardiograph

Within the context of this application the term complementary randomized data set refers to a data set that is complementary to the input set utilized to (fill in holes to) complete any cardiac data not available from the input set

Within the context of this application the term modeling data set refers to a data set of coefficients, constants, that are determined during the initialization procedure (prior to simulation) to determine provide system data based on input set and complementary set.

Within the context of this application the term monitoring input data set refers to a cardiac parameter data set comprising at least one or more and up to about seven cardiac parameters. Most preferably the monitoring input data set is preferably used to infer a plurality of monitored cardiac parameters.

Within the context of this application the term monitored cardiac parameter data set refers to the data set of cardiac parameters comprising a plurality of parameters that are determined with the personalized cardiac model that are abstracted/inferred/calculated/determined based on the monitoring input set.

Within the context of this application the term cardiac functions refers to the mathematical functions or derivations thereof that describe the hemodynamics of the cardiovascular system, the heart function and physiology, that are derived from a plurality of mathematical modeling functions for example including but not limited to elasticity equation derived from the generalized Hooke's law; passive Young moduli, active Young moduli; Euler equation, the Moens' equation, the law of conservation of mass and the law of conservation of energy.

Within the context of this application the term intra-cardiac cycle events refers to the 15 events and/or cases that collectively describe a single cardiac cycle, each of the 15 events and/or cases describe a snapshot of the cardiac cycle.

Within the context of this application the term functional cardiac workflow refer to the workflow described to determine d which of the 15 cardiac cycle events is representative of the available data set.

Within the context of this application the term right heart refers to the right side of the heart comprising the right ventricle and atrium.

Within the context of this application the term left heart refers to the left side of the heart comprising the left ventricle and atrium.

Within the context of this application the following symbols and/or acronyms may be used throughout the application body:

RA right atrium;

RV right ventricle;

LA left atrium;

LV left ventricle;

P pericardium;

Pa pulmonary artery;

L1 virtual pulmonary arteries;

L2 virtual pulmonary capillaries;

L3 virtual pulmonary veins;

Pv pulmonary vein;

Ao aorta;

B1 systemic arteries;

B2 systemic capillaries;

B3 systemic veins;

Vc vena cava.

Tr tricuspid valve

Mt mitral valve;

PLA Pressure left atrium;

PLV Pressure left ventricle;

PRA Pressure right atrium;

PRV Pressure right ventricle;

PAo Pressure in aorta;

PPa pressure pulmonary artery;

Ipred_LA Left atrial repolarization-depolarization timing

Ipred_LV Left ventricle repolarization-depolarization timing

Ipred_RA Right atrial repolarization-depolarization timing

Ipred_RV Right ventricle repolarization-depolarization timing;

Ea active Young's modulus

Ep passive Young's modulus

Most preferably in the first phase a cardiac hemodynamic model is abstracted relative to a primary set including a plurality of cardiac parameters wherein a cardiac hemodynamic model is abstracted to fit and accurately reflect a plurality of cardiac parameters. Most preferably the primary data set includes an input set of measured cardiac parameters, a complementary randomized data set, and a modeling data set.

Most preferably the personalized cardiac model is abstracted with a cardiac hemodynamic model abstractor and/or builder and/or simulator that most preferably attempts to build and/or abstract an accurate personalized cardiac model that accurately reflects and/or recreates the input data set of a plurality of cardiac parameters.

Most preferably the quality of an abstracted cardiac hemodynamic model is evaluated based on its adherence and/or ability to recreate the input data set of a plurality of cardiac parameters. Most preferably the cardiac hemodynamic model is evaluated in an evaluation process that evaluates the abstracted model by determining a penalty score for the abstracted cardiac model. Most preferably the penalty is determined based on the model's ability to predict the input set of a plurality of cardiac parameters. Optionally and preferably the penalty is evaluated relative to a penalty threshold level, if the penalty is below the threshold the abstracted mode) may be accepted, if the penalty score is above a threshold value the abstracted model is rejected and the process to abstract a new model is commenced.

Most preferably the primary data set is formed by initially obtaining the input set of measured cardiac parameters and building on that the complementary randomized data set followed by the modeling data set.

Most preferably the input set is a measured data set most preferably by way of image analysis and/or direct measurements. Optionally the input data set is provided by optional image processing techniques as is known in the art for example including but not limited to ultrasound, Doppler ultrasound, echocardiogram, angiogram, CT, MRI, PET, the like or any combination thereof.

Most preferably the complementary randomized data set is a system generated data set of cardiac parameters that is complementary to the input data set, including cardiac parameters that are not available and/or found in the input set. Most preferably the complementary data set comprises parameters that are provided with randomized values within a given (logical) data range based on the type of parameter and expected values and/or and within a given standard value range. Most preferably the complementary data set is generated and/or randomized by the abstractor. Most preferably after initial values are randomized by the abstractor, the system checks the validity of the abstracted complementary data set. Optionally the validity check is provided according to a rule based and/or logical hierarchy relative to the generated parameter. For example, internal diameter of a cardiac chamber is not larger than an external diameter of the same cardiac chamber.

Most preferably the modeling data set comprises parameters, coefficients, constants and the like mathematical data required to utilize the cardiac functions that are associated with the individual 15 events of the cardiac cycle. Optionally and most preferably the modeling data set is determined by the cardiac hemodynamic model abstractor and is determined during an initialization process based on the input data set and more preferably based on both the input set and complementary data set.

Most preferably the primary data set comprises a plurality cardiac parameters, most preferably as identified in Table 1 below:

TABLE 1
Cardiac Parameters description, data set and associated event

| Description | Data Set | Event Type |
|---|---|---|
| The internal radius of the non-deformed (empty) left ventricle | input or | intra-cycle |
| | complimentary | events |
| The external radius of the non-deformed (empty) | input or | intra-cycle |
| left ventricle | complimentary | events |
| The internal radius of the non-deformed (empty) | input or | intra-cycle |
| right ventricle | complimentary | events |
| The external radius of the non-deformed (empty) | input or | intra-cycle |
| right ventricle | complimentary | events |
| The left-atrial-and-pulmonary-vein blood density | input or | intra-cycle |
| | complimentary | events |
| The internal radius of the non-deformed (empty) | input or | intra-cycle |
| left atrium | complimentary | events |
| The external radius of the non-deformed (empty) | input or | intra-cycle |
| left atrium | complimentary | events |
| The right-atrial-and-vena-cava blood density | input or | intra-cycle |
| | complimentary | events |
| The internal radius of the non-deformed (empty) | input or | intra-cycle |
| right atrium | complimentary | events |
| The external radius of the non-deformed (empty) | input or | intra-cycle |
| right atrium | complimentary | events |
| The (internal) radius of the non-deformed | input or | intra-cycle |
| (empty) aorta | complimentary | events |
| The thickness of the non-deformed (empty) aorta | input or | intra-cycle |
| | complimentary | events |
| The length of aorta | input or | intra-cycle |
| | complimentary | events |
| The factor determining the pressure - effective | input or | intra-cycle |
| Young modulus relationship for aorta | complimentary | events |
| The (internal) radius of the non-deformed | input or | intra-cycle |
| (empty) vena cava | complimentary | events |
| The thickness of the non-deformed (empty) vena | input or | intra-cycle |
| cava | complimentary | events |
| The length of vena cava | input or | intra-cycle |
| | complimentary | events |
| The (internal) radius of the non-deformed | input or | intra-cycle |
| (empty) pulmonary artery | complimentary | events |
| The thickness of the non-deformed (empty) | input or | intra-cycle |
| pulmonary artery | complimentary | events |
| The length of pulmonary artery | input or | intra-cycle |
| | complimentary | events |
| The factor determining the pressure - effective | input or | intra-cycle |
| Young modulus relationship for Pa | complimentary | events |
| The (internal) radius of the non-deformed | input or | intra-cycle |
| (empty) pulmonary vein | complimentary | events |
| The thickness of the non-deformed (empty) | input or | intra-cycle |
| pulmonary vein | complimentary | events |
| The length of pulmonary vein | input or | intra-cycle |
| | complimentary | events |
| The (internal) radius of the non-deformed | input or | intra-cycle |
| (empty) L1 | complimentary | events |
| The (internal) radius of the non-deformed | input or | intra-cycle |
| (empty) L2 | complimentary | events |
| The thickness of the non-deformed (empty) L1 | input or | intra-cycle |
| | complimentary | events |
| The thickness of the non-deformed (empty) L2 | input or | intra-cycle |
| | complimentary | events |
| The thickness of the non-deformed (empty) L3 | input or | intra-cycle |
| | complimentary | events |
| The length of L1 | input or | intra-cycle |
| | complimentary | events |
| The length of L2 | input or | intra-cycle |
| | complimentary | events |
| The length of L3 | input or | intra-cycle |
| | complimentary | events |
| The density of blood in L1 | input or | intra-cycle |
| | complimentary | events |
| The density of blood in L2 | input or | intra-cycle |
| | complimentary | events |
| The density of blood in L3 | input or | intra-cycle |
| | complimentary | events |
| The viscosity-related resistance coefficient of the | input or | intra-cycle |
| blood flow in L1 | complimentary | events |
| The viscosity-related resistance coefficient of the | input or | intra-cycle |
| blood flow in L2 | complimentary | events |
| The viscosity-related resistance coefficient of the | input or | intra-cycle |
| blood flow in L3 | complimentary | events |
| The average radius of the non-deformed (empty) | input or | intra-cycle |
| system arteries | complimentary | events |
| The average thickness of the non-deformed | input or | intra-cycle |
| (empty) system arteries | complimentary | events |
| The average length of system arteries | input or | intra-cycle |
| | complimentary | events |
| The density of blood in system arteries | input or | intra-cycle |
| | complimentary | events |
| The viscosity-related resistance coefficient of the | input or | intra-cycle |
| blood flow in system arteries | complimentary | events |
| The average radius of the non-deformed (empty) | input or | intra-cycle |
| system capillaries | complimentary | events |
| The average thickness of the non-deformed | input or | intra-cycle |
| (empty) system capillaries | complimentary | events |
| The average length of system capillaries | input or | intra-cycle |
| | complimentary | events |
| The density of blood in system capillaries | input or | intra-cycle |
| | complimentary | events |
| The viscosity-related resistance coefficient of the | input or | intra-cycle |
| blood flow in system capillaries | complimentary | events |
| The average thickness of the non-deformed | input or | intra-cycle |
| (empty) system veins | complimentary | events |
| The average length of system veins | input or | intra-cycle |
| | complimentary | events |
| The density of blood in system veins | input or | intra-cycle |
| | complimentary | events |
| The viscosity-related resistance coefficient of the | modeling | intra-cycle |
| blood flow in system veins | | events |
| The minimal possible value and amplitude of the | modeling | intra-cycle |
| left atrial active Young modulus | | events |
| | modeling | intra-cycle |
| | | events |
| The minimal possible value and amplitude of the | modeling | intra-cycle |
| right atrial active Young modulus | | events |
| | modeling | intra-cycle |
| | | events |
| The minimal possible value and amplitude of the | modeling | intra-cycle |
| left ventricular active Young modulus | | events |
| | modeling | intra-cycle |
| | | events |
| The minimal possible value and amplitude of the | modeling | intra-cycle |
| right ventricular active Young modulus | | events |
| | modeling | intra-cycle |
| | | events |
| The minimal possible value, amplitude and | modeling | intra-cycle |
| exponential growth coefficients of the left-atrial | | events |
| passive Young modulus with respect to internal | modeling | intra-cycle |
| volume, wall thickness and pressure | | events |
| | modeling | intra-cycle |
| | | events |
| | modeling | intra-cycle |
| | | events |
| | modeling | intra-cycle |
| | | events |
| The minimal possible value, amplitude and | modeling | intra-cycle |
| exponential growth coefficients of the right-atrial | | events |
| passive Young modulus with respect to internal | modeling | intra-cycle |
| volume, wall thickness and pressure | | events |
| | modeling | intra-cycle |
| | | events |
| | modeling | intra-cycle |
| | | events |
| | modeling | intra-cycle |
| | | events |
| The minimal possible value, amplitude and | modeling | intra-cycle |
| exponential growth coefficients of the left- | | events |
| ventricular passive Young modulus with respect | modeling | intra-cycle |
| to internal volume, wall thickness and pressure | | events |
| | modeling | intra-cycle |
| | | events |
| | modeling | intra-cycle |
| | | events |
| | modeling | intra-cycle |
| | | events |
| The minimal possible value, amplitude and | modeling | intra-cycle |
| exponential growth coefficients of the right- | | events |
| ventricular passive Young modulus with respect | | |
| to internal volume, wall thickness and pressure | | |
| The Poisson coefficient of the right atrial wall | modeling | intra-cycle |
| material | | events |
| The Poisson coefficient of the left atrial wall | modeling | intra-cycle |
| material | | events |
| The Poisson coefficient of the right ventricular | modeling | intra-cycle |
| wall material | | events |
| The Poisson coefficient of the left ventricular | modeling | intra-cycle |
| wall material | | events |
| The mitral valve radius | modeling | intra-cycle |
| | | events |
| The tricuspid valve radius | modeling | intra-cycle |
| | | events |
| The mitral valve opening radius | modeling | intra-cycle |
| | | events |
| The tricuspid valve opening radius | modeling | intra-cycle |
| | | events |
| The systolic peak-shift related coefficient of the | modeling | intra-cycle |
| right-atrial Eₐ | | events |
| The systolic peak-shift related coefficient of the | modeling | intra-cycle |
| left-atrial Eₐ | | events |
| The systolic peak-shift related coefficient of the | modeling | intra-cycle |
| right-ventricular Eₐ | | events |
| The systolic peak-shift related coefficient of the | modeling | intra-cycle |
| left-ventricular Eₐ | | events |
| The diastolic hollow-shift related coefficient of | modeling | intra-cycle |
| the right-atrial Eₐ | | events |
| The diastolic hollow-shift related coefficient of | modeling | intra-cycle |
| the left-atrial Eₐ | | events |
| The diastolic hollow-shift related coefficient of | modeling | intra-cycle |
| the right-ventricular Eₐ | | events |
| The diastolic hollow-shift related coefficient of | modeling | intra-cycle |
| the left-ventricular Eₐ | | events |
| The systolic rise-related coefficient of the right- | modeling | intra-cycle |
| atrial Eₐ | | events |
| The systolic rise-related coefficient of the left- | modeling | intra-cycle |
| atrial Eₐ | | events |
| The systolic rise-related coefficient of the right- | modeling | intra-cycle |
| ventricular Eₐ | | events |
| The systolic rise-related coefficient of the left- | modeling | intra-cycle |
| ventricular Eₐ | | events |
| The diastolic descent-related coefficient of the | modeling | intra-cycle |
| right-atrial Eₐ | | events |
| The diastolic descent-related coefficient of the | modeling | intra-cycle |
| left-atrial Eₐ | | events |
| The diastolic descent-related coefficient of the | modeling | intra-cycle |
| right-ventricular Eₐ | | events |
| The diastolic descent-related coefficient of the | modeling | intra-cycle |
| left-ventricular Eₐ | | events |
| The wall thickness of the non-deformed (empty) | modeling | intra-cycle |
| pericardial chamber | | events |
| The Young modulus of the pericardial wall | modeling | intra-cycle |
| material | | events |
| The parameter determining an initial value of | modeling | intra-cycle |
| p_L1 | | events |
| The parameter determining an initial value of | modeling | intra-cycle |
| p_L2 | | events |
| The parameter determining an initial value of | modeling | intra-cycle |
| p_L3 | | events |
| The parameter determining an initial value of | modeling | intra-cycle |
| p_B1 | | events |
| The parameter determining an initial value of | modeling | intra-cycle |
| p_B2 | | events |
| The parameter determining an initial value of | modeling | intra-cycle |
| p_B3 | | events |
| The ratio of external to internal radius of the non- | modeling | intra-cycle |
| deformed (empty) left ventricle: k = R₂/R₁ | | events |
| The connected elasticity matrix elements | modeling | intra-cycle |
| (functions of k): | | events |
| | modeling | intra-cycle |
| | | events |
| | modeling | intra-cycle |
| | | events |
| | modeling | intra-cycle |
| | | events |
| The ratio of external to internal radius of the non- | modeling | intra-cycle |
| deformed (empty) right ventricle: k = R₂/R₁ | | events |
| The connected elasticity matrix elements | modeling | intra-cycle |
| (functions of k): | | events |
| | modeling | intra-cycle |
| | | events |
| | modeling | intra-cycle |
| | | events |
| | modeling | intra-cycle |
| | | events |
| The ratio of external to internal radius of the non- | modeling | intra-cycle |
| deformed (empty) left atrium: k = R₂/R₁ | | events |
| The connected elasticity matrix elements | modeling | intra-cycle |
| (functions of k): | | events |
| | modeling | intra-cycle |
| | | events |
| | modeling | intra-cycle |
| | | events |
| | modeling | intra-cycle |
| | | events |
| The ratio of external to internal radius of the non- | modeling | intra-cycle |
| deformed (empty) right atrium: k = R₂/R₁ | | events |
| The connected elasticity matrix elements | modeling | intra-cycle |
| (functions of k): | | events |
| | modeling | intra-cycle |
| | | events |
| | modeling | intra-cycle |
| | | events |
| | modeling | intra-cycle |
| | | events |
| The internal radius of the non-deformed (empty) | modeling | intra-cycle |
| pericardial chamber: | | events |
| The external radius of the non-deformed (empty) | modeling | intra-cycle |
| pericardial chamber: R₂ = R₁ + h | | events |
| The ratio of external to internal radius of the non- | modeling | intra-cycle |
| deformed (empty) pericardial chamber: k = | | events |
| R₂/R₁ | | |
| The connected elasticity matrix elements | modeling | intra-cycle |
| (functions of k): | | events |
| | modeling | intra-cycle |
| | | events |
| | modeling | intra-cycle |
| | | events |
| | modeling | intra-cycle |
| | | events |
| The left-ventricular wall's value | modeling | intra-cycle |
| | | events |
| The right-ventricular wall's value | modeling | intra-cycle |
| | | events |
| The left-atrial wall's value | modeling | intra-cycle |
| | | events |
| The right-atrial wall's value | modeling | intra-cycle |
| | | events |
| LA amplitude | modeling | intra-cycle |
| | | events |
| RA ample | modeling | intra-cycle |
| | | events |
| LV ampl | modeling | intra-cycle |
| | | events |
| RV ampl | modeling | intra-cycle |
| | | events |
| D₁(RA) | modeling | intra-cycle |
| | | events |
| D₁(LA) | modeling | intra-cycle |
| | | events |
| D₁(RV) | modeling | intra-cycle |
| | | events |
| D₁(LV) | modeling | intra-cycle |
| | | events |
| Eₐ₂(LA) | modeling | intra-cycle |
| | | events |
| Eₐ₄(LA) | modeling | intra-cycle |
| | | events |
| Eₐ₂(RA) | modeling | intra-cycle |
| | | events |
| Eₐ₄(RA) | modeling | intra-cycle |
| | | events |
| Eₐ₂(LV) | modeling | intra-cycle |
| | | events |
| Eₐ₄(LV) | modeling | intra-cycle |
| | | events |
| Eₐ₂(RV) | modeling | intra-cycle |
| | | events |
| Eₐ₄(RV) | modeling | intra-cycle |
| | | events |
| The Young modulus of the aortic wall referred to | modeling | intra-cycle |
| zero pressure | | events |
| The effective Young modulus of the vena cava | modeling | intra-cycle |
| wall: | | events |
| | modeling | intra-cycle |
| | | events |
| The vena cava absolute pressure wave | modeling | intra-cycle |
| propagation velocity: | | events |
| | modeling | intra-cycle |
| | | events |
| The Young modulus of the pulmonary-arterial | modeling | intra-cycle |
| wall referred to zero pressure | | events |
| The effective Young modulus of the pulmonary | modeling | intra-cycle |
| vein wall: | | events |
| The pulmonary vein absolute pressure wave | modeling | intra-cycle |
| propagation velocity: | | events |
| The average effective Young modulus of the | modeling | intra-cycle |
| system capillaries' walls: | | events |
| The average effective Young modulus of the | modeling | intra-cycle |
| system veins' walls: | | events |
| The average effective Young modulus of L2 | modeling | intra-cycle |
| walls: | | events |
| | modeling | intra-cycle |
| | | events |
| The average effective Young modulus of L3 | modeling | intra-cycle |
| walls: | | events |
| Right Atrium Constant | modeling | intra-cycle |
| | | events |
| Left Atrium Constant | modeling | intra-cycle |
| | | events |
| Right Ventricle Constant | modeling | intra-cycle |
| | | events |
| Left Ventricle Constant | modeling | intra-cycle |
| | | events |
| The moment of the end of left-ventricular systole | modeling | intra-cycle |
| | | events |
| The moment of the end of left-ventricular diastole | modeling | intra-cycle |
| | | events |
| The moment of the end of left-ventricular next | modeling | intra-cycle |
| diastole | | events |
| The moment of the end of right-ventricular | modeling | intra-cycle |
| systole | | events |
| The moment of the end of right-ventricular | modeling | intra-cycle |
| diastole | | events |
| The moment of the end of right-ventricular next | modeling | intra-cycle |
| diastole | | events |
| The moment of the end of left-atrial systole | modeling | intra-cycle |
| | | events |
| The moment of the start of left-ventricular filling | modeling | intra-cycle |
| | | events |
| The moment of the start of left-atrial next systole | modeling | intra-cycle |
| | | events |
| The moment of the end of left-atrial next systole | modeling | intra-cycle |
| | | events |
| The moment of the end of right-atrial systole | modeling | intra-cycle |
| | | events |
| The moment of the start of right-ventricular | modeling | intra-cycle |
| filling | | events |
| The moment of the start of right-atrial next | modeling | intra-cycle |
| systole | | events |
| The moment of the end of right-atrial next systole | modeling | intra-cycle |
| | | events |
| Vector of the end indices of RV's phases | modeling | intra-cycle |
| | | events |
| Vector of the end indices of LV's phases | modeling | intra-cycle |
| | | events |
| Vector of the end indices of RA's phases | modeling | intra-cycle |
| | | events |
| Vector of the end indices of LA's phases | modeling | intra-cycle |
| | | events |
| Vector of indices of the timing points basic for | modeling | intra-cycle |
| the determination of Ea_RA | | events |
| Vector of indices of the timing points basic for | modeling | intra-cycle |
| the determination of Ea_LA | | events |
| Vector of indices of the timing points basic for | modeling | intra-cycle |
| the determination of Ea_RV | | events |
| Vector of indices of the timing points basic for | modeling | intra-cycle |
| the determination of Ea_LV | | events |
| The left-ventricular blood pressure | modeling | intra-cycle |
| | | events |
| The active Young modulus of the left-ventricular | modeling | intra-cycle |
| wall: | | events |
| | modeling | intra-cycle |
| | | events |
| The passive Young modulus of the left- | modeling | intra-cycle |
| ventricular wall: | | events |
| The effective Young modulus of the left- | modeling | intra-cycle |
| ventricular wall; E = Eₐ + Eₚ | | events |
| The absolute deformation-related increment of | modeling | intra-cycle |
| internal left-ventricular radius | | events |
| The absolute deformation-related increments of | modeling | intra-cycle |
| external left-ventricular radius | | events |
| The right-ventricular blood pressure | modeling | intra-cycle |
| | | events |
| The active Young modulus of the right- | modeling | intra-cycle |
| ventricular wall: | | events |
| The passive Young modulus of the right- | modeling | intra-cycle |
| ventricular wall: | | events |
| The effective Young modulus of the right- | modeling | intra-cycle |
| ventricular wall: E = Eₐ + Eₚ | | events |
| The absolute deformation-related increment of | modeling | intra-cycle |
| internal right-ventricular radius | | events |
| The absolute deformation-related increment of | modeling | intra-cycle |
| external right-ventricular radius | | events |
| The left-atrial blood pressure | modeling | intra-cycle |
| | | events |
| The active Young modulus of the left-atrial wall: | modeling | intra-cycle |
| | | events |
| The passive Young modulus of the left-atrial | modeling | intra-cycle |
| wall: | | events |
| The effective Young modulus of the left-atrial | modeling | intra-cycle |
| wall: E = Eₐ + Eₚ | | events |
| The absolute deformation-related increment of | modeling | intra-cycle |
| internal left-atrial radius | | events |
| The absolute deformation-related increment of | modeling | intra-cycle |
| external left-atrial radius | | events |
| The flow velocity on mitral valve | modeling | intra-cycle |
| | | events |
| The absolute left-atrial pressure wave | modeling | intra-cycle |
| propagation velocity | | events |
| The right-atrial blood pressure | modeling | intra-cycle |
| | | events |
| The active Young modulus of the right-atrial | modeling | intra-cycle |
| wall: | | events |
| | modeling | intra-cycle |
| | | events |
| The passive Young modulus of the right-atrial | modeling | intra-cycle |
| wall: | | events |
| The effective Young modulus of the right-atrial | modeling | intra-cycle |
| wall: E = Eₐ + Eₚ | | events |
| The absolute deformation-related increment of | modeling | intra-cycle |
| internal right-atrial radius | | events |
| The absolute deformation-related increment of | modeling | intra-cycle |
| external right-atrial radius | | events |
| The flow velocity on tricuspid valve | modeling | intra-cycle |
| | | events |
| The absolute right-atrial pressure wave | modeling | intra-cycle |
| propagation velocity | | events |
| The aortic blood pressure | modeling | intra-cycle |
| | | events |
| The density of blood fluid in aorta | modeling | intra-cycle |
| | | events |
| The axial blood flow velocity in aorta | modeling | intra-cycle |
| | | events |
| The increment of the volume flow from aorta to | modeling | intra-cycle |
| B1 | | events |
| The aortic absolute pressure wave propagation | modeling | intra-cycle |
| velocity: | | events |
| The effective Young modulus of the aortic wall: | modeling | intra-cycle |
| | | events |
| The absolute deformation-related increment of | modeling | intra-cycle |
| the aortic radius | | events |
| The vena cava blood pressure | modeling | intra-cycle |
| | | events |
| The axial blood flow velocity in vena cava | modeling | intra-cycle |
| | | events |
| The absolute deformation-related increment of | modeling | intra-cycle |
| the vena cava radius | | events |
| The increment of the volume flow from B3 to | modeling | intra-cycle |
| vena cava | | events |
| The pulmonary artery blood pressure | modeling | intra-cycle |
| | | events |
| The density of blood fluid in pulmonary artery | modeling | intra-cycle |
| | | events |
| The axial blood flow velocity in pulmonary artery | modeling | intra-cycle |
| | | events |
| The axial blood flow velocity in pulmonary vein | modeling | intra-cycle |
| | | events |
| The increment of the volume flow from RV to Pa | modeling | intra-cycle |
| | | events |
| The increment of the volume flow from Pa to L1 | modeling | intra-cycle |
| | | events |
| The increment of the volume flow from L1 to L2 | modeling | intra-cycle |
| | | events |
| The increment of the volume flow from L2 to L3 | modeling | intra-cycle |
| | | events |
| The increment of the volume flow from L3 to Pv | modeling | intra-cycle |
| | | events |
| The increment of the volume flow from Pv to LA | modeling | intra-cycle |
| | | events |
| The Pa absolute pressure wave propagation | modeling | intra-cycle |
| velocity: | | events |
| | modeling | intra-cycle |
| | | events |
| The effective Young modulus of the Pa wall: | modeling | intra-cycle |
| | | events |
| The absolute deformation-related increment of | modeling | intra-cycle |
| the pulmonary artery radius | | events |
| The pulmonary vein blood pressure | modeling | intra-cycle |
| | | events |
| The absolute deformation-related increment of | modeling | intra-cycle |
| the pulmonary vein radius | | events |
| The blood pressure in L1 | modeling | intra-cycle |
| | | events |
| The average effective Young modulus of L1 | modeling | intra-cycle |
| walls | | events |
| The absolute deformation-related increment of | modeling | intra-cycle |
| the L1 radius | | events |
| The L1 resistance: | modeling | intra-cycle |
| | | events |
| The blood pressure in L2 | modeling | intra-cycle |
| | | events |
| The absolute deformation-related increment of | modeling | intra-cycle |
| the L2 radius | | events |
| The L2 resistance: | modeling | intra-cycle |
| | | events |
| The blood pressure in L3 | modeling | intra-cycle |
| | | events |
| The (internal) radius of the non-deformed | modeling | intra-cycle |
| (empty) L3 | | events |
| The absolute deformation-related increment of | modeling | intra-cycle |
| the L3 radius | | events |
| The L3 resistance: | modeling | intra-cycle |
| | | events |
| The increment of the volume flow from LV to Ao | modeling | intra-cycle |
| | | events |
| The increment of the volume flow from Ao to B1 | modeling | intra-cycle |
| | | events |
| The average system arterial blood pressure | modeling | intra-cycle |
| | | events |
| The average effective Young modulus of the | modeling | intra-cycle |
| system arteries' walls | | events |
| The average absolute deformation-related | modeling | intra-cycle |
| increment of system arteries | | events |
| The average resistance of system arteries: | modeling | intra-cycle |
| | | events |
| The increment of the volume flow from system | modeling | intra-cycle |
| arteries to capillaries | | events |
| The average system capillary blood pressure | modeling | intra-cycle |
| | | events |
| The average absolute deformation-related | modeling | intra-cycle |
| increment of system capillaries | | events |
| The average resistance of system arteries: | modeling | intra-cycle |
| | | events |
| The increment of the volume flow from system | modeling | intra-cycle |
| capillaries to veins | | events |
| The average system venous blood pressure | modeling | intra-cycle |
| | | events |
| The average radius of the non-deformed (empty) | modeling | intra-cycle |
| system veins | | events |
| The average absolute deformation-related | modeling | intra-cycle |
| increment of system veins | | events |
| The average resistance of system veins: | modeling | intra-cycle |
| | | events |
| The increment of the volume flow from B3 to Vc | modeling | intra-cycle |
| | | events |
| The increment of the volume flow from Vc to RA | modeling | intra-cycle |
| | | events |
| The intro-pericardial pressure | modeling | intra-cycle |
| | | events |
| The absolute deformation-related increment of | modeling | intra-cycle |
| internal pericardial radius | | events |
| The absolute deformation-related increments of | modeling | intra-cycle |
| external pericardial radius | | events |
| The internal volume of (deformed) left- | modeling | intra-cycle |
| ventricular | | events |
| The internal volume of (deformed) right ventricle | modeling | intra-cycle |
| | | events |
| The internal volume of (deformed) left atrium | modeling | intra-cycle |
| | | events |
| The internal volume of (deformed) right atrium | modeling | ultra-cycle |
| | | events |
| The internal volume of (deformed) aorta | modeling | intra-cycle |
| | | events |
| The internal volume of (deformed) vena cava | modeling | intra-cycle |
| | | events |
| The internal volume of (deformed) pulmonary | modeling | intra-cycle |
| artery | | events |
| The internal volume of (deformed) pulmonary | modeling | intra-cycle |
| vein | | events |
| The internal volume of (deformed) L1 | modeling | intra-cycle |
| | | events |
| The internal volume of (deformed) L2 | modeling | intra-cycle |
| | | events |
| The internal volume of (deformed) L3 | modeling | intra-cycle |
| | | events |
| The internal volume of (deformed) B1 | modeling | intra-cycle |
| | | events |
| The internal volume of (deformed) B2 | modeling | intra-cycle |
| | | events |
| The internal volume of (deformed) B3 | modeling | intra-cycle |
| | | events |
| The parameter of the function determining | modeling | inter-cycle |
| b_ampl (LV) via the left-ventricular EDV | | events |
| The parameter of the function determining | modeling | inter-cycle |
| b_ampl (RV) via the right-ventricular EDV | | events |
| The parameter of the function determining | modeling | inter-cycle |
| b_ampl (LA) via the left-atrial pre-systolic | | events |
| volume | | |
| The parameter of the function determining | modeling | inter-cycle |
| b_ampl (RA) via the right-atrial pre-systolic | | events |
| volume | | |
| The parameter of the function determining | modeling | inter-cycle |
| b_D1(LV) via the left-ventricular EDV | | events |
| The parameter of the function determining | modeling | inter-cycle |
| b_D1(RV) via the right-ventricular EDV | | events |
| The parameter of the function determining | modeling | inter-cycle |
| b_D1(LA) via the left-atrial EDV | | events |
| The parameter of the function determining | modeling | inter-cycle |
| b_D1(RA) via the right-atrial EDV | | events |
| The parameter of the function determining | modeling | inter-cycle |
| b_R(B3) via the blood pressure in Ao or Pa | | events |
| The parameter of the function determining | modeling | inter-cycle |
| b_R(L3) via the blood pressure in Ao or Pa | | events |
| The parameter of the function determining | modeling | inter-cycle |
| b_E(B1) via the blood pressure in B2 | | events |
| The parameter of the function determining | modeling | inter-cycle |
| b_E(L1) via the blood pressure in L2 | | events |
| The parameter of the function determining | modeling | inter-cycle |
| b_dt(LV) via the blood pressure in L2 | | events |
| The parameter of the function determining | modeling | inter-cycle |
| b_dt(RV) via the blood pressure in B2 | | events |
| The coefficient regulating ampl (LV) via the | modeling | inter-cycle |
| left-ventricular EDV | | events |
| The coefficient regulating ampl (RV) via the | modeling | inter-cycle |
| right-ventricular EDV | | events |
| The coefficient regulating ampl (LA) via the left- | modeling | inter-cycle |
| atrial pre-systolic volume | | events |
| The coefficient regulating ampl (RA) via the | modeling | inter-cycle |
| right-atrial pre-systolic volume | | events |
| The coefficient regulating D₁(LV) via the left- | modeling | inter-cycle |
| ventricular EDV | | events |
| The coefficient regulating D₁(RV) via the right- | modeling | inter-cycle |
| ventricular EDV | | events |
| The coefficient regulating D₁(LA) via the left- | modeling | inter-cycle |
| atrial EDV | | events |
| The coefficient regulating D₁(RA) via the right- | modeling | inter-cycle |
| atrial EDV | | events |
| The coefficient regulating R_{B3} via the blood | modeling | inter-cycle |
| pressure in Ao or Pa | | events |
| The coefficient regulating R_{L3} via the blood | modeling | inter-cycle |
| pressure in Ao or Pa | | events |
| The coefficient regulating E_{B1} via the blood | modeling | inter-cycle |
| pressure in B2 | | events |
| The coefficient regulating E_{L1} via the blood | modeling | inter-cycle |
| pressure in L2 | | events |
| The coefficient regulating LV diastolic duration | modeling | inter-cycle |
| via the blood pressure in L2 | | events |
| The coefficient regulating RV diastolic duration | modeling | inter-cycle |
| via the blood pressure in B2 | | events |
| Heart Rate | ECG input | monitoring |
| PQ duration | ECG input | monitoring |
| QRS duration | ECG input | monitoring |
| ST duration | ECG input | monitoring |
| T wave duration | ECG input | monitoring |
| P wave duration | ECG input | monitoring |
| The factual delay between systole of RA and RA | ECG input | monitoring |
| The minimal possible delay between systole of | ECG input | monitoring |
| RA and RA | | |
| The delay between systole of RV and LV | ECG input | monitoring |
| The standard Pwave duration | ECG input | monitoring |

Most preferably the input set comprises a plurality of measured cardiac parameters. Optionally and preferably a plurality of cardiac parameters forming at least a portion of the input set may be obtained by way of image processing and/or analysis of cardiac imagery and/or data. For example, image processing based parameter may be provided by an imaging device for example including but not limited to ultrasound, Doppler ultrasound, echocardiogram, angiogram, CT, MRI, PET, the like or any combination thereof.

Optionally a plurality of cardiac parameter may be obtained for the input set from optional non-imagery medical devices for example including but is not limited to sphygmomanometer, blood pressure device, direct catheterization, implanted device, electrocardiograph (‘ECG’ or ‘EKG’), laboratory testing, blood works parameters the like, or any combination thereof.

Within the context of this application the term implanted devices may refer to any implant that provides data about any structure and/or anatomy of the cardiovascular system. Optionally implanted devices may be implanted about, coupled to, and/or in association therewith whether direct and/or indirect, wired and/or wireless with any structure and/or anatomy of the cardiopulmonary system for example including the heart, lungs, any cells, any neurons, any arteries, any veins, any vessels, ganglions, or the like anatomical structures.

Optionally and preferably the input set of a plurality of cardiac parameters provided by image processing techniques, for example including but not limited to the echocardiogram parameters relating to the Aorta, Pulmonary Artery, Heart left side (ventricle and atrium), Heart right side (ventricle and atrium). Optionally and preferably the input set comprises the following data parameters when derived from echocardiogram: Aortic lumen during cardio cycle, Ao valve opening and closing time, blood flow velocity in Aorta, blood flow velocity on Ao valve, Pulmonary Artery Lumen during cardio cycle, blood flow velocity in Pulmonary Artery, blood flow velocity on PA valve, Systolic and Diastolic Left ventricle Diameter, Mitral valve opening and closing time; Left ventricle volume during cardio cycle; Left Atrium diameters; Left Atrium Area maximal; Left Atrium area minimal; left ventricle systolic wall thickness systolic; left ventricle diastolic wall thickness; blood flow velocity through mitral valve; cardio cycle timing; Systolic right ventricle Long diameter; Diastolic right ventricle Long diameter; Systolic right ventricle short diameter; Diastolic right ventricle short diameter; Right Atrium diameter; Right Atrium maximal Area; Right atrium minimal area; blood flow velocity through tricuspid valve; the like or any combination thereof.

Most preferably following the formation of the primary data set, the cardiac model abstractor initiates the process for abstracting the personalized cardiac model based the data of the primary set. Most preferably the hemodynamic model is abstracted by a plurality of iterations and evaluation of a plurality of cardiac functions that depict an individual cardiac cycle in an event by event basis (case by case basis) where individual cardiac events are evaluated. Most preferably evaluation of a plurality of cardiac parameters from the perspective of the cardiac cycle events provide for abstracting a personalized cardiac hemodynamic model with increased resolution, therefore providing a more accurate account of the cardiac hemodynamic of an individual that is preferably highly correlated to the functionality of the heart.

Most preferably the cardiac hemodynamic model is abstracted by evaluating the primary data set through a functional cardiac workflow that mirrors the events of a single cardiac cycle therein closely modeling relative to the workflow of the cardiac cycle over a single cardiac cycle, rather than the generalized entire heart anatomical model utilized to date.

Most preferably the abstractor evaluates the data available in the primary data set to determine which of the 15 cardiac cycle events it is represented with and is reflected by the primary data set values.

Most preferably the cardiac workflow of a single cardiac cycle comprises 15 cases and/or cardiac events reflecting the various events in a single cardiac cycle. Most preferably each of the 15 cardiac cycle cases individually identify an instantaneous snap shot of the cardiac cycle. The 15 cardiac cycle cases collectively account for a single full cardiac cycle.

Most preferably each of the 15 cardiac cases forming the workflow are associated with a plurality of cardiac functions modeling the specific cardiac cycle event Therein most preferably each of the 15 cardiac events is associated with a plurality of cardiac functions that describe the hearts functionality at the specific and/or instantaneous event within the cardiac cycle.

Most preferably the 15 cardiac cycle events comprise and account for the following events of the cardiac cycle, as depicted in the table 2 below:

TABLE 2
Intra-Cardiac Cycle Events

| Right/Left | Atrial | Isovolumic | | Isovolumic | |
|---|---|---|---|---|---|
| Sides | Systole | contraction | Ejection | relaxation | Filling |
| Atrial Systole | Event 1 | Event 3 | Reject | Reject | Event 14 |
| Isovolumic | Event 2 | Event 4 | Event 6 | Reject | Reject |
| contraction | | | | | |
| Ejection | Reject | Event 5 | Event 7 | Event 9 | Reject |
| Isovolumic | Reject | Reject | Event 8 | Event 10 | Event 12 |
| relaxation | | | | | |
| Filling | event 15 | Reject | Reject | Event 11 | Event 13 |

Most preferably the 15 cardiac cycle events and/or cases are depicted below: Both hearts (left side and right side) are in atrial systole; left heart is in atrial systole, the right heart is in isovolumic contraction; the right heart is in atrial systole, the left heart is on isovolumic contraction; Both hearts are in isovolumic contraction; The left heart is in isovolumic contraction, the right heart is in ejection phase; The right is in isovolumetric contraction, the left heart is in ejection phase; Both hearts are in ejection phases; the left heart is in ejection phase, the right heart is in isovolumic relaxation; the right heart is in ejection phase, the left heart is in isovolumic relaxation; both hearts are in isovolumic relaxation; the left heart is in isovolumic relaxation, the right heart is in filling phase; the right heart is in isovolumic relaxation, the left heart is in filling phase; Both hearts are in filling phases; the left heart is in filling phase, the right heart is in atrial systole; the right heart is in filling phase, the left heart is in atrial systole.

Most preferably each of the 15 cases reflecting the cardiac cycle events is associated with and evaluates a particular set of cardiac functions reiterating the specific cardiac activity. Optionally and preferably each of the 15 cases may be associated with a plurality of cardiac functions that are derived from and/or include the following equations as is known in the art: elasticity equation derived from the generalized Hooke's law; passive Young moduli, active Young moduli; Euler, equation, the Moens' equation, the law of conservation of mass and the law of conservation of energy, derivations thereof, the like, or any combination thereof.

Optionally and preferably the cardiac equations are associated with a particular case and/or event is outlined in Table 3 below:

TABLE 3

| Case/ | |
|---|---|
| Event | Function Description |
| 1-15 | Determination of the Young modules |
| 6-8 | Determination of the parameters corresponding to chains |
| | RV → Pa and LV → Ao |
| 3-5 | Determination of the ventricular parameters on isovolumic |
| | contraction |
| 9-11 | Determination of the ventricular parameters on isovolumic |
| | relaxation |
| 12-14 | Determination of the parameters corresponding to chains |
| | Vc → RA → RV and Pv → LA → LV on rapid or reduced |
| | ventricular filling |
| 1 | Newton's Method applied to integrate/balance between |
| | equations and parameters utilized in specific case 1 |
| 2 | Newton's Method applied to integrate/balance between |
| | equations and parameters utilized in specific case 2 |
| 3 | Newton's Method applied to integrate/balance between |
| | equations and parameters utilized in specific case 3 |
| 4, 10 | Newton's Method applied to integrate/balance between |
| | equations and parameters utilized in specific case 4 and 10 |
| 5, 9 | Newton's Method applied to integrate/balance between |
| | equations and parameters utilized in specific case 5 and 9 |
| 6, 8 | Newton's Method applied to integrate/balance between |
| | equations and parameters utilized in specific case 6 and 8 |
| 7 | Newton's Method applied to integrate/balance between |
| | equations and parameters utilized in specific case 7 |
| 12 | Newton's Method applied to integrate/balance between |
| | equations and parameters utilized in specific case 12 |
| 14 | Newton's Method applied to integrate/balance between |
| | equations and parameters utilized in specific case 14 |
| 11 | Newton's Method applied to integrate/balance between |
| | equations and parameters utilized in specific case 11 |
| 15 | Newton's Method applied to integrate/balance between |
| | equations and parameters utilized in specific case 15 |
| 13 | Newton's Method applied to integrate/balance between |
| | equations and parameters utilized in specific case 13 |
| 1-15 | Determination of the parameters corresponding to blood |
| | circulation PA and AO |
| 5-7 | Determination of the parameters corresponding to chains |
| | Vc → RA and Pv → LA |
| 8 | Determination of the parameters corresponding to chains |
| | Vc → RA and Pv → LA |
| 1, 2, 15 | Determination of the parameters corresponding to chains |
| | Pv → LA → LV on atrial systole |
| 2, 4, 6 | Determination of the ventricular parameters on isovolumic |
| | contraction and relaxation |
| 8, 10, 12 | Determination of the ventricular parameters on isovolumic |
| | contraction and relaxation |
| 5, 7, 9 | Determination of the parameters corresponding to chains |
| | RV → Pa and LV → Ao |
| 11, 13, 15 | Determination of the parameters corresponding to chains |
| | Vc → RA → RV and Pv → LA → LV on rapid or reduced |
| | ventricular filling |
| 2, 8, 10, 12 | Determination of the parameters corresponding to chains |
| | Vc → RA and Pv → LA |
| 1, 3, 14 | Determination of the parameters corresponding to chains |
| | Vc → RA → RV on atrial systole |
| 1-15 | Determination of the parameters corresponding to blood |
| | circulation Pa → L1 → L2 → L3 → Pv |
| 1-15 | Determination of the parameters corresponding to blood |
| | circulation Ao → B1 → B2 → B3 → Vc |
| Inter-cycle | pressure-related regulation for Ao and PA |
| Inter-cycle | pre-systolic volume-related regulation |
| Inter-cycle | repolarization-depolarization timing of cardiac chambers |

Most preferably the initial cardiac cycle event (Sn=1 . . . 15, n=0) may be determined by evaluating the primary data set with respect to cardiac pressure in the different cardiac chambers. Most preferably the initialization process, evaluates the cardiac chamber pressure relative to one another. Most preferably during the initialization, the abstractor determines the volume flow increments as well as the pressure ratio between cardiac chambers, for example including but not limited to PLA/PLV; PRA/PRV; PLV/PAo; PRV/PPa; Ipred_LA; Ipred_LV; Ipred_RA; Ipred_RV. Based on the relative pressure evaluation the abstractor determines which cardiac cycle event (1-15) is defined by the primary data set,

Most preferably following the initial cardiac cycle event evaluation (S=Sn, n={1 . . . 15}) the abstractor evaluates the respective cardiac functions associated with the given cardiac cycle event (S=Sn), based on the primary data set. Most preferably following the evaluation of cardiac functions associated with the given cardiac cycle event (S=Sn), the parameters forming the primary parameter set are updated.

Next the updated primary parameters set is evaluated to determine the next cardiac cycle event (Sₙ₊₁) which may be the same event (n=n), the previous sequential event (n=n−1) or the next sequential event (n=n+1). Optionally the evaluation process may reveal that the cardiac cycle event remains unchanged where (S=Sₙ) or that the primary set parameters indicate that the parameters progressed to the next sequential cardiac cycle event (S=Sₙ₊₁=Sₙ+1, n={1 . . . 15}) or regressed to the previous sequential cardiac cycle event. For example, if the initial event was event 1 (n=1) the next event may be any event defined by n=15, n=1 or n=2.

Most preferably the reiterative evaluation process of cardiac cycle event (1-15) and updating the primary parameters set according to the state associated cardiac parameters, as described above, continues for at least a single full cardiac cycle, identified by cycling through all 15 events at least once, in a sequential manner from the initial stage, therein ensuring at least one full cycle. Most preferably evaluation of cardiac cycle events may be undertaken at a frequency of 10 ms.

Next once a full cycle has been performed, the primary set is evaluated with additional inter-cycle cardiac functions. Most preferably the inter-cycle cardiac functions model hemodynamics regulation processes. Optionally and preferably these inter-cycle cardiac functions provided to re-evaluate and adjust the primary set as necessary for stroke volume parameters, most preferably accounting for pressure-related regulation most preferably evaluated for the respective 4 cardiac chambers. The inter-cardiac cycle functions are preferably associated with inter-cardiac cycle events based on the status of the cardiac chambers for example including but not limited to after filling and before atrial systole and/or after atrial systole before isovolumic contraction on either of the right side or left side.

Following the evaluation of the inter-cycle cardiac functions and the primary data set is updated accordingly and/or adjusted the cardiac cycle state is evaluated and continuously adjusted as described above.

Most preferably this reiterative evaluation of the cardiac functions relative to the cardiac cycle events continues for a plurality of cycles. Optionally and preferably the number of cycles simulation may be defined by a user and/or system according to resources, the like or any combination thereof.

Optionally and most preferably at least 3 cycles are simulated before an initial model stability evaluation process is undertaken, to check for stable state. Optionally and most preferably stable state is determined by comparing all pressure hemodynamics parameters characteristics associated with the all cardiac chambers particularly left ventricle and right ventricle, and the end diastolic pressure cardiovascular parameters. Optionally, if the pending model has not reached a stable state the system reverts and continues simulating up to about to 30 cardiac cycles, until the model achieves stable state.

Optionally if a stable state is not reached within a 30 cycle period the systems reverts to the initialization stages where the primary parameter set is reset. Most preferably the reset primary data set is reset by forming a new complementary data set and thereafter re-evaluate the modeling data set forming a new primary data set to abstract a new model.

Most preferably following simulation of a plurality of cycles the abstracted module is evaluated for its accuracy relative to a penalty score. Optionally and most preferably the penalty score is determined relative to the primary data set and in particular the input parameter set and their behavior over time relative to expected and logical norms.

Most preferably with each iteration the primary data set, about its randomized data set portion is adjusted so as to optimize the results. For example the cross-entropy method may be utilized to optimize the randomized data set portion of the primary data set, there in sequentially improving the system's performance to reduce the penalty score. The process is continued until an acceptable, below threshold, penalty value is obtained by the abstractor.

Most preferably once a personalized cardiac model is abstracted it may be utilized for monitoring cardiac parameters. Most preferably monitoring cardiac parameters provides for utilizing at least one and up to seven monitoring input parameters to infer a plurality of cardiac parameters with the cardiac hemodynamic model.

The cardiac hemodynamic model preferably comprises and defines a plurality of parameters, for example including but not limited to the parameters outline din table 4 below:

TABLE 4
The parameters defining the cardiac hemodynamic model

| Parameter Name | Data Set Origin | Description |
|---|---|---|
| LV_EDV | input or complementary | Estimated left ventricle end diastolic volume |
| LV_ESV | input or complementary | Estimated left ventricle end systolic volume |
| Septal_wall_thick_ | input or complementary | Estimated left ventricle wall thickness in |
| LV_S | | systole |
| Estim_RV_EDV | input or complementary | Estimated right ventricle end diastolic volume |
| Lateral_wall_thick_ | input or complementary | Estimated right ventricle wall thickness in |
| RV_max | | systole |
| RA_diam | input or complementary | Right atrial diameter |
| Lateral_wall_thick_ | input or complementary | Estimated right atrial wall thickness in systole |
| RA_S | | |
| Lateral_wall_thick_ | input or complementary | Estimated right atrial wall thickness in |
| RA_D | | diastole |
| LA_diam | input or complementary | Right atrial diameter |
| Septal_wall_thick_ | input or complementary | Estimated minimal left atrial wall thickness |
| LA_min | | |
| Septal_wall_thick_ | input or complementary | Estimated maximal left atrial wall thickness in |
| LA_max | | systole |
| Ascending_Ao | input or complementary | Diameter of Ascending Aorta |
| PA_dimension | input or complementary | Pulmonary artery dimension |
| p_Ao1 | input or complementary | Aortic Pressure (diastolic) |
| p_Ao2 | input or complementary | Aortic Pressure (systolic) |
| R_(LV)1[1] | complementary | The internal radius of the non-deformed |
| | | (empty) left ventricle |
| R_2(LV) | complementary | The external radius of the non-deformed |
| | | (empty) left ventricle |
| R_1(RV) | complementary | The internal radius of the non-deformed |
| | | (empty) right ventricle |
| R_2(RV) | complementary | The external radius of the non-deformed |
| | | (empty) right ventricle |
| R_1(LA) | complementary | The internal radius of the non-deformed |
| | | (empty) left atrium |
| R_2(LA) | complementary | The external radius of the non-deformed |
| | | (empty) left atrium |
| R_1(RA) | complementary | The internal radius of the non-deformed |
| | | (empty) right atrium |
| R_2(RA) | complementary | The external radius of the non-deformed |
| | | (empty) right atrium |
| R_Ao | complementary | The (internal) radius of the non-deformed |
| | | (empty) aorta |
| R_(Vc) | complementary | The (internal) radius of the non-deformed |
| | | (empty) vena cava |
| R_Pa | complementary | The (internal) radius of the non-deformed |
| | | (empty) pulmonary artery |
| R_(Pv) | complementary | The (internal) radius of the non-deformed |
| | | (empty) pulmonary vein |
| R_L1 | complementary | The (internal) radius of the non-deformed |
| | | (empty) L1 |
| R_L2 | complementary | The (internal) radius of the non-deformed |
| | | (empty) L2 |
| R_L3 | complementary | The (internal) radius of the non-deformed |
| | | (empty) L3 |
| mu_L1 | complementary | The viscosity-related resistance coefficient of |
| | | the blood flow in L1 |
| mu_L2 | complementary | The viscosity-related resistance coefficient of |
| | | the blood flow in L2 |
| mu_L3 | complementary | The viscosity-related resistance coefficient of |
| | | the blood flow in L3 |
| R_B1 | complementary | The average radius of the non-deformed |
| | | (empty) system arteries |
| mu_B1 | complementary | The viscosity-related resistance coefficient of |
| | | the blood flow in system arteries |
| R_B2 | complementary | The average radius of the non-deformed |
| | | (empty) system capillaries |
| mu_B2 | complementary | The viscosity-related resistance coefficient of |
| | | the blood flow in system capillaries |
| R_B3 | complementary | The average radius of the non-deformed |
| | | (empty) system veins |
| mu_B3 | complementary | The viscosity-related resistance coefficient of |
| | | the blood flow in system veins |
| E0_(LA) | complementary | The minimal possible value of the left atrial |
| | | active Young modulus |
| ampl0(LA) | complementary | The minimal possible amplitude of the left |
| | | atrial active Young modulus |
| E0_(RA) | complementary | The minimal possible value of the right atrial |
| | | active Young modulus |
| ampl0(RA) | complementary | The minimal possible amplitude of the right |
| | | atrial active Young modulus |
| E0_(LV) | complementry | The minimal possible value of the left |
| | | ventricular active Young modulus |
| ampl0(LV) | complementary | The minimal possible amplitude of the left |
| | | ventricular active Young modulus |
| E0_(RV) | complementary | The minimal possible value of the right |
| | | ventricular active Young modulus |
| ampl0(RV) | complementary | The minimal possible amplitude of the right |
| | | ventricular active Young modulus |
| ampl_p(LA) | complementary | The minimal possible amplitude of the left- |
| | | atrial passive Young modulus |
| regul_flow(LA) | complementary | Correction factor of flow during filling phase |
| | | due to left ventricular not sphericity |
| Ch_Ep(LA) | complementary | coefficients of the left-atrial passive Young |
| | | modulus with respect to wall thickness |
| ampl_p(RA) | complementary | The minimal possible amplitude of the right- |
| | | atrial passive Young modulus |
| regul_flow(RA) | complementary | Correction factor of flow during filling phase |
| | | due to right ventricular not sphericity |
| Ch_Ep(RA) | complementary | coefficients of the right-atrial passive Young |
| | | modulus with respect to wall thickness |
| ampl_p(LV) | complementary | The minimal possible amplitude of the left- |
| | | ventricle passive Young modulus |
| regul_veloc(LA) | complementary | Correction factor of flow during Atrial systole |
| | | phase due to left ventricular not sphericity |
| Ch_Ep(LV) | complementary | coefficients of the left-ventricle passive |
| | | Young modulus with respect to wall thickness |
| ampl_p(RV) | complementary | The minimal possible amplitude of the right- |
| | | ventricle passive Young modulus |
| regul_veloc(RA) | complementary | Correction factor of flow during Atrial systole |
| | | phase due to right ventricular not sphericity |
| Ch_Ep(RV) | complementary | coefficients of the right-ventricle passive |
| | | Young modulus with respect to wall thickness |
| n1_Ea(RA) | complementary | The systolic peak-shift related coefficient of |
| | | the right-atrial Ea |
| n1_Ea(LA) | complementary | The systolic peak-shift related coefficient of |
| | | the left-atrial Ea |
| n1_Ea(RV) | complementary | The systolic peak-shift related coefficient of |
| | | the right-ventricular Ea |
| n1_Ea(LV) | complementary | The systolic peak-shift related coefficient of |
| | | the left-ventricular Ea |
| n2_Ea(RA) | complementary | The diastolic hollow-shift related coefficient |
| | | of the right-atrial Ea |
| n2_Ea(LA) | complementary | The diastolic hollow-shift related coefficient |
| | | of the left-atrial Ea |
| n2_Ea(LV) | complementary | The diastolic hollow-shift related coefficient |
| | | of the right-ventricular Ea |
| n2_Ea(RV) | complementary | The diastolic hollow-shift related coefficient |
| | | of the left-ventricular Ea |
| D1_Ea0(RA) | complementary | The systolic rise-related coefficient of the |
| | | right-atrial Ea |
| D1_Ea0(LA) | complementary | The systolic rise-related coefficient of the left- |
| | | atrial Ea |
| D1_Ea0(RV) | complementary | The systolic rise-related coefficient of the |
| | | right-ventricular Ea |
| D1_Ea0(LV) | complementary | The systolic rise-related coefficient of the left- |
| | | ventricular Ea |
| D2_Ea(RA) | complementary | The diastolic descent-related coefficient or the |
| | | right-atrial Ea |
| D2_Ea(LA) | complementary | The diastolic descent-related coefficient of the |
| | | left-atrial Ea |
| D2_Ea(LV) | complementary | The diastolic descent-related coefficient of the |
| | | right-ventricular Ea |
| D2_Ea(RV) | complementary | The diastolic descent-related coefficient of the |
| | | left-ventricular Ea |
| dp_L1 | complementary | The parameter determining an initial value of |
| | | p_L1 |
| dp_L2 | complementary | The parameter determining an initial value of |
| | | p_L2 |
| dp_L3 | complementary | The parameter determining an initial value of |
| | | p_L3 |
| dp_B1 | complementary | The parameter determining an initial value of |
| | | p_B1 |
| dp_B2 | complementary | The parameter determining an initial value of |
| | | p_B2 |
| dp_B3 | complementary | The parameter determining an initial value of |
| | | p_B3 |
| R_valve(LA) | complementary | The mitral valve maximal radius during filling |
| R_valve(RA) | complementary | The tricuspid valve maximal radius during |
| | | filling |
| R_Tr_AS | complementary | Tricuspid valve maximal opening radius |
| | | during Atrial systole |
| R_Mt_AS | complementary | Mitral valve maximal opening radius during |
| | | Atrial systole |
| h_P | complementary | The wall thickness of the non-deformed |
| | | (empty) pericardial chamber |
| E_P | complementary | The Young modulus of the pericardial wall |
| | | material |
| del_L1 | complementary | The average deformation of pulmonary |
| | | arteries in ″0″ point |
| del_L2 | complementary | The average deformation of pulmonary |
| | | capillaries in ″0″ point |
| de_L3 | complementary | The average deformation of pulmonary veins |
| | | in ″0″point |
| del_B1 | complementary | The average deformation of systemic arteries |
| | | in ″0″ point |
| del_B2 | complementary | The average deformation of systemic |
| | | capillaries in 0″ point |
| del_B3 | complementary | The average deformation of systemic veins in |
| | | ″0″ point |
| VBC | complementary | Volume of blood circulation |
| length_L1 | complementary | The length of L1 |
| length_L2 | complementary | The length of L2 |
| length_L3 | complementary | The length of L3 |
| length_B1 | complementary | The average length of system arteries |
| length_B2 | complementary | The average length of system capillaries |
| length_B3 | complementary | The average length of system veins |
| length_Ao | complementary | The length of aorta |
| length_Vc | complementary | The length of vena cava |
| length_Pa | complementary | The length of pulmonary artery |
| length_Pv | complementary | The length pulmonary vein |
| Vc_dimension | complementary | Vena Cava dimension |
| Pv_dimension | complementary | Pulmonary vein dimension |
| St_p(RA) | complementary | coefficient of dependency of the passive |
| | | Young's modulus from the RA wall |
| | | hypertrophy |
| St_p(LA) | complementary | coefficient of dependency of the passive |
| | | Young's modulus from the LA wall |
| | | hypertrophy |
| St_p(RV) | complementary | coefficient of dependency of the passive |
| | | Young's modulus from the RV wall |
| | | hypertrophy |
| St_p(LV) | complementary | coefficient of dependency of the pasive |
| | | Young's modulus from the LV wall |
| | | hypertrophy |
| Ten(RA) | complementary | coefficient of dependency of the RA intra- |
| | | myocardial tension from the RA wall |
| | | hypertrophy |
| Ten(LA) | complementary | coefficient of dependency of the LA intra- |
| | | myocardial tension from the LA wall |
| | | hypertrophy |
| Ten(RV) | complementary | coefficient of dependency of the RV intra- |
| | | myocardial tension from the RV wall |
| | | hypertrophy |
| Ten(LV) | complementary | coefficient of dependency of the LV intra- |
| | | myocardial tension from the LV wall |
| | | hypertrophy |
| k_gam(RA) | complementary | coefficient of dependency of the RA intra- |
| | | myocardial tension from the RA active |
| | | Young's modulus amplitude |
| k_gam(LA) | complementary | coefficient of dependency of the LA intra- |
| | | myocardial tension from the LA active |
| | | Young's modulus amplitude |
| k_gam(RV) | complementary | coefficient of dependency of the RV intra- |
| | | myocardial tension from the RV active |
| | | Young's modulus amplitude |
| k_gam(LV) | complementary | coefficient of dependency of the LV intra- |
| | | myocardial tension from the LV active |
| | | Young's modulus amplitude |
| dt_cs_RV | complementary | time delay between end of right ventricle |
| | | myocardial cells repolarization and beginning |
| | | Isovolumic relaxation |
| dt_es_LV | complementary | time delay between end of left ventricle |
| | | myocardial cells repolarization and beginning |
| | | Isovolumic relaxation |
| del_Ao | Initial calculation | The average deformation of Aorta in ″0″ |
| | | point |
| del_Pa | Initial calculation | The average deformation of Pulmonary |
| | | Artery in ″0″ point |
| del_LA_1 | Initial calculation | The average deformation of Left Atrium in |
| | | ″0″ point |
| del_LV_1 | Initial calculation | The average deformation of Left ventricle in |
| | | ″0″ point |
| p_LV | Initial calculation | Estimated left ventricle pressure in ″0″ point |
| p_RV | Initial calculation | Estimated right ventricle pressure in ″0″ point |
| p_LA | Initial calculation | Estimated left atrial pressure in ″0″ point |
| p_RA | Initial calculation | Estimated right atrial pressure in ″0″ point |
| p_Ao | Initial calculation | Estimated aortic pressure in ″0″ point |
| p_Pa | Initial calculation | Estimated pulmonary artery pressure in ″0″ |
| | | point |

Most preferably during and the second phase where the personalized cardiac model abstracted in phase 1 is utilized to monitor cardiac parameters based on at least one or more and up to about seven input monitoring cardiac parameters. Optionally and preferably during monitoring an input of a minimal set of cardiac parameters for example at least one and up to about seven cardiac parameters may be used to generate a full set of cardiac parameters as an output monitoring data set.

Optionally and preferably the input of minimal set of monitoring cardiac input parameters may for example be selected from the group consisting on Left ventricle volume, Left ventricle volume and PA flow velocity monitoring, Aortic flow velocity and Tricuspid valve flow velocity monitoring, Aortic flow velocity and Mitral valve flow velocity monitoring, Right Ventricle Pressure monitoring, Pulmonary Artery Pressure monitoring, Left Ventricle Pressure monitoring.

Most preferably the hemodynamic parameter output as a result of monitoring may for example include but is not limited to at least one and more preferably a plurality of output parameters selected from the group for example including but not limited to: Left Ventricle Pressure; Right Ventricle Pressure; Left Atrium Pressure; Right Atrium Pressure; Pressure in Aorta; Pressure in Pulmonary Artery; Pressure drop in the arterial, capillary and venous components of the systemic circulation; Pressure drop in the arterial, capillary and venous components of the, pulmonary circulation; Left Ventricle volume; Right Ventricle volume; Left Atrium volume; Right Atrium volume; Aortic Lumen; PA Lumen; Left ventricle Wall thickness; Right ventricle Wall thickness; Left Ventricle Intra-myocardial tensions and stresses; Right Ventricle Intra-myocardial tensions and stresses; Blood flow velocity in Aorta; Blood flow velocity in Pulmonary Artery; Blood flow passage through the Aortic valve; Blood flow passage through the PA valve; Blood flow passage through the Mitral valve; Blood flow passage through the Tricuspid valve; Systemic circulation Resistance; Pulmonary circulation Resistance; Right Ventricular pressure-volume relation; Left Ventricular pressure-volume relation; Pericardial pressure; Pericardial volume, the like or any combination thereof.

Most preferably during monitoring the input monitoring data set is simulated with the abstracted model, where most preferably a monitoring primary data set is defined including the monitoring input data set and the modeling parameter constants defining the personalized cardiac model abstracted and identified in phase 1.

Most preferably the monitoring data set is then simulated in a similar manner to that utilized during the abstraction process where most preferably the primary data set is fed into the model where the various cardiac modules are evaluated relative to the 15 cardiac events as previously described. Most preferably during the simulation process the primary monitoring data set is updated where parameters and data are added to provide a plurality of cardiac parameters not part of the monitoring input set to form an output monitoring data set.

Optionally and preferably the monitoring simulation process continues for the length of data available in the monitoring input set. Therefore most preferably the number of simulated cardiac cycles available during monitoring is directly determined by the number of cardiac cycles available in the monitoring input data set.

Optionally and preferably the monitoring may be performed offline relative to recorded input imagery monitoring data, as previously described. Optionally monitoring may be performed online, substantially in real time during active real time monitoring of an individual, with imagery data, most preferably to provide output monitoring parameters data set substantially in real time.

An optional embodiment of the present invention provides for a further third phase in abstracting and monitoring the personalized cardiac model, most preferably an optional third phase provided to account for anatomical cardiac remodeling where the abstracted model is updated at given time intervals, and/or following cardiac events to account for any cardiac remodeling occurring over time and/or due to cardiac events.

Optionally the personalized cardiac model abstracted during the first phase, as described above, may be updated over time, for example at given and controllable time intervals. Optionally the re-evaluation time interval may for example be from about three months up to about one year from the end of abstracting the model. Optionally re-evaluation time interval may be about 3 months, more preferably about 6 months, optionally and preferably about 9 months and most preferably about 12 months. Optionally and preferably such re-evaluation is provided to account for any anatomical cardiac remodeling that may have taken place of the give time period.

Optionally phase three comprising model re-evaluation may be provided following any one or more events for example including but not limited to medical intervention, change in personalized drug profile, patient profile, disease profile, physiological events, biological events, anatomical events, events that directly or indirectly affect the functionality of the cardiovascular system, the like events, or any combination thereof. For example, the model may be re-evaluated following cardiac events for example including but not limited to an infarction, stroke, seizure, heart attack, surgery, placement of a stent, angioplasty, minimally invasive surgery, valve replacement surgery, any sensed anatomical changes for example wall thickening, the like or any combination thereof.

Unless otherwise defined the various embodiment of the present invention may be provided to an end user in a plurality of formats, platforms, and may be outputted to at least one of a computer readable memory, a computer display device, a printout, a computer on a network or a user.

The processes associated with some of the present embodiments may be executed by programmable equipment, such as computers. Software that may cause programmable equipment to execute the processes may be stored in any storage device, such as, for example, a computer system (non-volatile) memory, disk-on-key, flash memory device, an optical disk, magnetic tape, or magnetic disk. Furthermore, some of the processes may be programmed when the computer system is manufactured or via a computer-readable medium at a later date. Such a medium may include any of the forms listed above with respect to storage devices and may further include, for example, a carrier wave modulated, or otherwise manipulated, to convey instructions that can be read, demodulated/decoded and executed by a computer.

It can be appreciated, for example, that some process aspects described herein may be performed, in certain embodiments, using instructions stored on a computer-readable medium or media that direct a computer system to perform the process aspects. A computer-readable medium can include, for example, memory devices such as diskettes, compact discs of both read-only and read/write varieties, optical disk drives, and hard disk drives, flash-memory devices, disk-on-key, or the like. A computer-readable medium can also include memory storage that can be physical, virtual, permanent, temporary, semi-permanent and/or semi-temporary. A computer-readable medium can further include one or more data signals transmitted on one or more carrier waves.

A “computer” or “computer system” may be, for example, a wireless or wire-line variety of a microcomputer, minicomputer, laptop, personal data assistant (PDA), wireless e-mail device, cellular phone, pager, processor, or any other programmable device, which devices may be capable of configuration for transmitting and receiving data over a network. Computer devices disclosed herein can include memory for storing certain software applications used in obtaining, processing and communicating data. It can be appreciated that such memory can be internal or external. The memory can also include any means for storing software, including a hard disk, an optical disk, floppy disk, ROM (read only memory), RAM (random access memory), PROM (programmable ROM), EEPROM (electrically erasable PROM), flash memory, and other computer-readable media.

It is to be understood that the figures and descriptions of the embodiments of the present invention have been simplified to illustrate elements that are relevant for a clear understanding of the present invention, while eliminating, for purposes of clarity, other elements. Those of ordinary skill in the art will recognize that these and other elements may be desirable. However, because such elements are well known in the art, and because they do not facilitate a better understanding of the present invention, a discussion of such elements is not provided herein.

Unless otherwise defined, all technical and scientific terms used herein have the same meaning as commonly understood by one of ordinary skill in the art to which this invention belongs. The materials, methods, and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an exemplary system according to the present invention;

FIG. 2 is an exemplary method according to the present invention for abstracting a personalized cardiac model and monitoring a plurality of cardiac parameters based on the personalized cardiac model;

FIG. 3 is an exemplary method according to the present invention, depicting the steps for simulating and abstracting a personalized cardiac model;

FIG. 4A is a schematic block diagram illustrating the system according to the present invention when abstracting an event based personalized cardiac hemodynamic model according to optional embodiments of the present invention;

FIG. 4B is a schematic block diagram illustrating the system according to the present invention when monitoring hemodynamic and cardiac parameters with an abstracted personalized cardiac hemodynamic model according to optional embodiments of the present invention;

FIG. 5 is a schematic block diagram showing greater details of the correlation between cardiac cycle events and cardiac function in abstracting and monitoring hemodynamic cardiac parameters;

FIG. 6 is an illustrative block diagram of the event evaluator according to optional embodiments of the present invention;

FIG. 7 is a flowchart of the event classifier according to optional embodiments of the present invention; and

FIGS. 8A-8D are expanded portions of the flowchart depicted in FIG. 7.

### DESCRIPTION OF THE EMBODIMENTS

The principles and operation of the present invention may be better understood with reference to the drawings and the accompanying description.

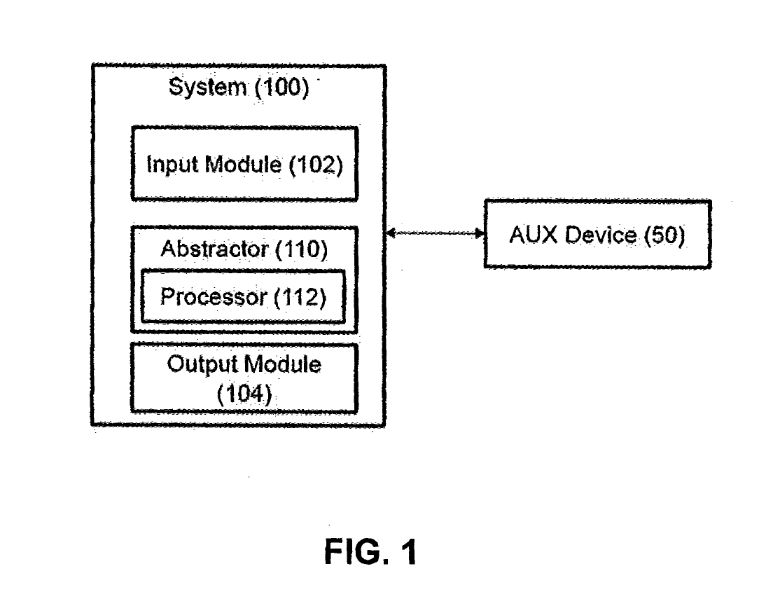

Referring now to the drawings, FIG. 1 is a schematic block diagram of an exemplary system 100 according to the present invention for abstracting a personalized cardiac model that may be utilized for monitoring a plurality of cardiac parameters. Most preferably system 100 comprises an input module 102, an output module 104 and an abstractor 110.

Optionally system 100 may associate and/or be functional with at least one or more auxiliary devices 50. Optionally auxiliary device may interface and/or communicate with input module 102 and/or output module 104.

Most preferably input module 102 provides for receiving and/or processing an input set of cardiac parameters and communicating the input set to abstractor 110 for further processing.

Optionally input module 102 may receive an input set of cardiac parameters from at least one or more external and/or auxiliary device 50. Optionally an auxiliary device 50 may be an offline device for transmitting data, for example including but not limited to a computer and/or server or the like.

Optionally auxiliary device 50 may be an online monitoring device for example including but not limited to ultrasound system, electrocardiogram, catheterization, imaginary data, imagery device, MRI, CT, PET or the like.

Optionally auxiliary device 50 may be provided in the form of a device capable of communicating with input module 102. For example communication may comprise auxiliary device 50 sending raw and/or processed data to input module 102 for further processing, according to optional methods of the present invention. For example, auxiliary device 50 may provide image processing data that is raw and/or processed that is provided to system 100 via input module 102 for abstracting a hemodynamic cardiac model 150. Optionally auxiliary device 50 may provide system 100 with a data set (input data set) for monitoring with the hemodynamic model 150. Optionally auxiliary device 50 may provide system 100 with the input data set and cardiac mode data set for monitoring a plurality of cardiac parameters. Optionally auxiliary device 50 may communicate to a cardiac hemodynamic model 150, abstracted according to the present invention for monitoring and/or evaluation.

Optionally auxiliary device 50 may for example include but is not limited to an image processing device, computer, server, a mobile communication device, a smartphone, an implanted device, a health care-giver system, health care-giver database, decision support system, echocardiograph, ultrasound, CT, MRI, PET, image processor, non-imagery measuring device, sensor, data storage device, online monitoring device, sphygmomanometer, blood pressure device, direct catheterization device, electronic devices, implanted device, electrocardiograph (‘ECG’ or ‘EKG’), laboratory testing device, blood works parameters, or the like.

Most preferably abstractor 110 provides for generating and/or abstracting a personalized cardiac model based on a primary set of cardiac parameters produced with input module 102, Most preferably abstractor 110 is characterized in that it facilitates generating a personalized cardiac model based on an evaluation of a plurality of cardiac cycle events wherein each cardiac cycle stage is associated with a plurality of cardiac functions that model the individual cardiac cycle state, Most preferably the cardiac cycle states reflect the various events during the cardiac cycle.

Most preferably abstractor 110 utilizes 15 cardiac cycle state selected from the group consisting of: both hearts are in atrial systole; left heart is in atrial systole, the right heart is in isovolumic contraction; the right heart is in atrial systole, the left heart is on isovolumic contraction; Both hearts are in isovolumic contraction; The left heart is in isovolumic contraction, the right heart is in ejection phase; The right is in isovolumetric contraction, the left heart is in ejection phase; Both hearts are in ejection phases; the left heart is in ejection phase, the right heart is in isovolumic relaxation; the right heart is in ejection phase, the left heart is in isovolumic relaxation; both hearts are in isovolumic relaxation; the left heart is in isovolumic relaxation, the right heart is in filling phase; the right heart is in isovolumic relaxation, the left heart is in filling phase; Both hearts are in filling phases; the left heart is in filling phase, the right heart is in atrial systole; the right heart is in filling phase, the left heart is in atrial systole.

Most preferably each cardiac cycle stage may be associated with a plurality of cardiac function selected from the group consisting of equations derived from and/or based on the following base equations: elasticity equation derived from the generalized Hooke's law; passive Young moduli, active Young moduli; Euler equation, the Moens' equation, the law of conservation of mass and the law of conservation of energy.

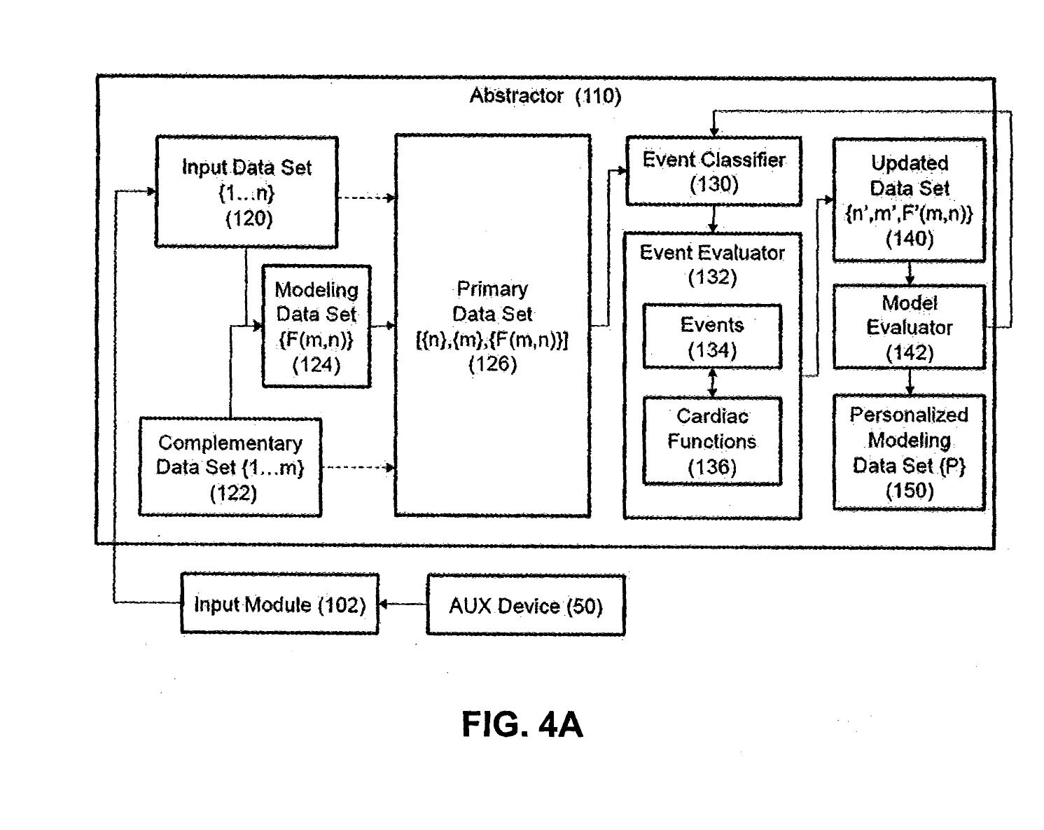

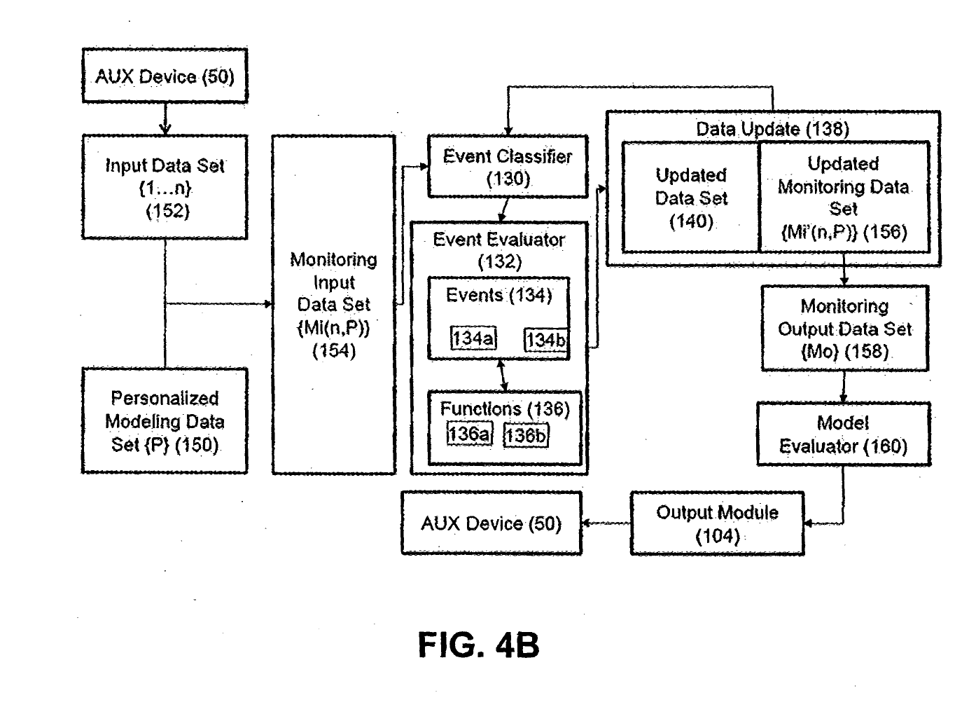

Most preferably abstractor 110 comprises a processor 112, shown in greater detail in FIG. 4A, that facilitates evaluating a plurality of cardiac parameters that are associated with individual cardiac cycle states, while abstracting the personalized cardiac model, according to the present invention.

Most preferably abstractor 110 further provides for monitoring cardiac parameters with the abstracted personalized cardiac model. Most preferably abstractor 110 processes and/or evaluates an input set of cardiac parameters comprising at least one and up to seven input cardiac parameters, communicated from input module 102, to produces a plurality of output parameters that are preferably communicated to output module 104.

Optionally and preferably the output cardiac parameters produced with abstractor 110 may be selected from the group consisting of Left Ventricle Pressure; Right Ventricle Pressure; Left Atrium Pressure; Right Atrium Pressure; Pressure in Aorta; Pressure in Pulmonary Artery; Pressure drop in the systemic circulation; Pressure drop in the arterial systemic circulation; Pressure drop in the capillary systemic circulation; Pressure drop in the venous components of the systemic circulation; Pressure drop in the pulmonary circulation; Pressure drop in the arterial pulmonary circulation; Pressure drop in the capillary pulmonary circulation; Pressure drop in the venous components of the pulmonary circulation; Left Ventricle volume; Right Ventricle volume; Left Atrium volume; Right Atrium volume; Aortic Lumen; PA Lumen; Left ventricle Wall thickness; Right ventricle Wall thickness; Left Ventricle Intra-myocardial tensions and stresses; Right Ventricle Intra-myocardial tensions and stresses; Blood flow velocity in Aorta; Blood flow velocity in Pulmonary Artery; Blood flow passage through the Aortic valve; Blood flow passage through the PA valve; Blood flow passage through the Mitral valve; Blood flow passage through the Tricuspid valve; Systemic circulation Resistance; Pulmonary circulation Resistance; Right Ventricular pressure-volume relation; Left Ventricular pressure-volume relation; Pericardial pressure; Pericardial volume, the like, in any combination thereof.

Most preferably output module 104 provides for communicating and displaying a set of output cardiac parameters following processing with abstractor 110.

Optionally output module 104 may communicate and/or exchange data with at least one or more external and/or auxiliary device 50, for example for further processing, displaying, printing, analysis, communicating an alarm state or the like. For example output module may communicate an output set of cardiac parameter to an optional auxiliary device 50.

Optionally output module 104 may communicate with an auxiliary device 50 wherein an alarm state is communicated to auxiliary device 50. Optionally output module 104 may communicate data to an auxiliary device 50 wherein the auxiliary device performs further processing to identify an alarm state.

Optionally system 100 may be utilized in a home setting by an end-user to abstract his/her own personalized cardiac hemodynamic model according to optional embodiments of the present invention,

Optionally system 100 may be utilized in a home setting by an end-user to monitor a plurality of cardiac parameters with a personalized cardiac hemodynamic model.

Optionally system 100 may be utilized in a home setting by an end-user to monitor a plurality of cardiac parameters with a personalized cardiac hemodynamic model abstracted according to optional embodiments of the present invention.

Optionally system 100 may be utilized in a hospital and/or clinic and/or care-giver setting by a trained physician and/or technician to abstract a personalized cardiac hemodynamic model according to optional embodiments of the present invention.

Optionally system 100 may be utilized in a hospital and/or clinic and/or care-giver setting by a trained physician and/or technician to monitor a plurality of cardiac parameters with a personalized cardiac hemodynamic model.

Optionally system 100 may be utilized in a hospital and/or clinic and/or care-giver setting by a trained physician and/or technician to monitor a plurality of cardiac parameters with a personalized cardiac hemodynamic model abstracted according to optional embodiments of the present invention.

Optionally monitoring in a hospital setting may be provided in essentially in real time wherein an input monitoring parameters are obtained and cardiac monitoring is provided according to optional methods of the present invention therein producing a plurality of cardiac monitoring parameters substantially in real time.

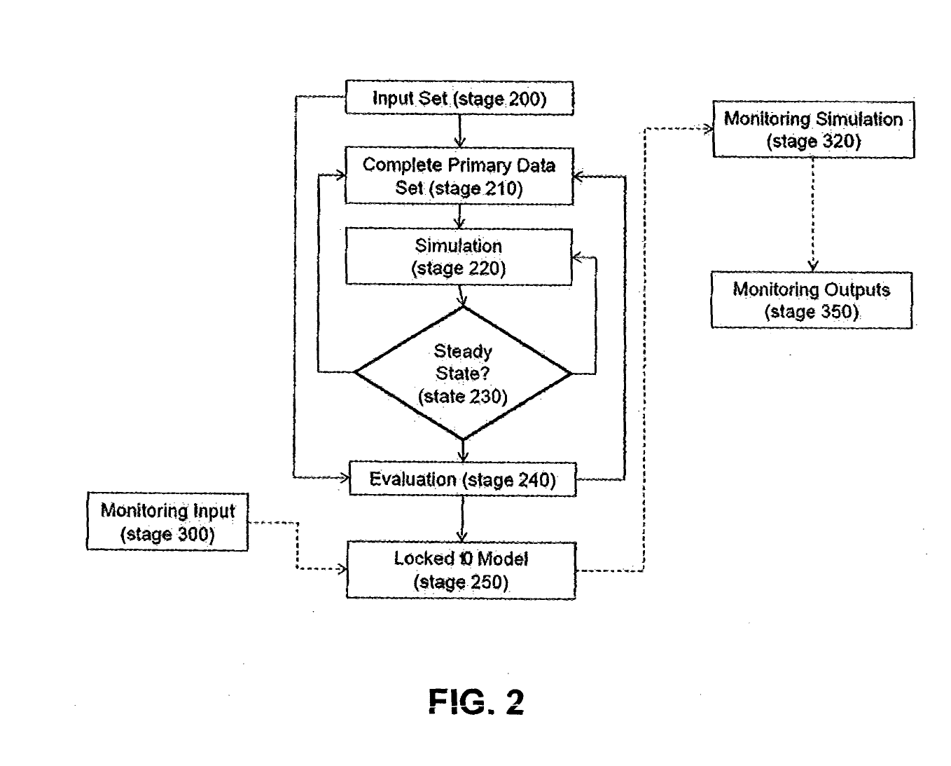

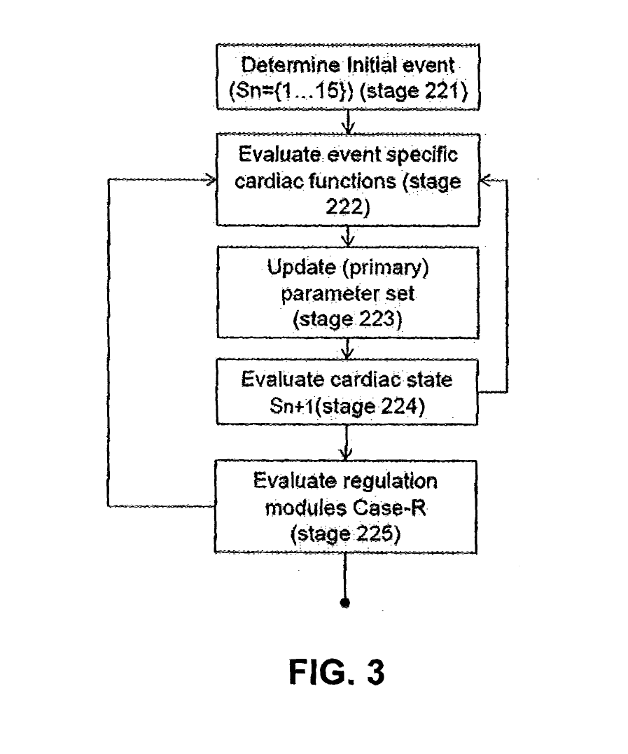

FIG. 2-3 show a flowchart of an exemplary method for abstracting a personalized cardiac hemodynamic model and for monitoring a plurality of cardiac parameters, according to the present invention. Most preferably the method may be rendered by system 100 depicted in FIG. 1, in particular abstractor 110, and further illustrated in greater detail in FIG. 4A-B. Optionally and preferably the method of the present invention may be practiced in a two phase process, the first phase provided for abstracting a personalized cardiac model and a second phase provided for monitoring cardiac parameters with the abstracted personalized cardiac model from the first phase.

Optionally a third phase may be utilized to update the abstracted model over time, for example re-evaluating the model at given time interval, or due to physiological events, that may bring about cardiac remodeling.

Most preferably the method of abstracting a personalized cardiac model starts in stage 200 where an input set of parameters comprising a plurality of cardiac parameters is measured. Optionally and most preferably the input data set is a measured data set most preferably obtained by way of image analysis and/or direct measurements. Optionally the input data set may be obtained with an auxiliary device 50 for example an imagery device for example including but not limited to an echocardiograph, ultrasound, CT, MRI, PET or the like device, for example as shown in FIG. 4A.

Most preferably the input set comprises a plurality of measured cardiac parameters. Optionally and preferably a plurality of cardiac parameters forming at least a portion of the input set 120 may be obtained by way of image processing and/or analysis of cardiac imagery and/or data, for example provided by input module 102 a depicted in FIGS. 1 and 4A. For example, image processing based parameter may be provided by an imaging device, in the form of an auxiliary device 50 and/or as part of input module 102, for example including but not limited to ultrasound, Doppler ultrasound, echocardiogram, angiogram, CT, MRI, PET, the like or any combination thereof.

Optionally a plurality of cardiac parameter may be obtained for the input set from optional non-imagery medical devices, optionally in the form of an auxiliary device associated with the system, for example including but is not limited to sphygmomanometer, blood pressure device, direct catheterization, implanted device, electrocardiograph (‘ECG’ or ‘EKG’), laboratory testing, blood works parameters the like, or any combination thereof. Optionally non-imagery medical devices may be provided in the form of auxiliary device 50 with input data that may be processed via input module 102, for example as shown in FIG. 4A.

Optionally and preferably input set 120 comprising of a plurality of cardiac parameters provided by image processing techniques, for example including but not limited to the echocardiogram parameters relating to the Aorta, Pulmonary Artery, Heart left side (ventricle and atrium), Heart right side (ventricle and atrium). Optionally the input set comprises a plurality of parameters selected from the following parameters for example including but not limited to; Aortic lumen during cardio cycle, Ao valve opening and closing time, blood flow velocity in Aorta, blood flow velocity on Ao valve, Pulmonary Artery Lumen during cardio cycle, blood flow velocity in Pulmonary Artery, blood flow velocity on PA valve, Systolic and Diastolic Left ventricle Diameter, Mitral valve opening and closing time; Left ventricle volume during cardio cycle; Left Atrium diameters; Left Atrium Area maximal; Left Atrium area minimal; left ventricle systolic wall thickness systolic; left ventricle diastolic wall thickness; blood flow velocity through mitral valve; cardio cycle timing; Systolic right ventricle Long diameter; Diastolic right ventricle Long diameter; Systolic right ventricle short diameter; Diastolic right ventricle short diameter; Right Atrium diameter; Right Atrium maximal Area; Right atrium minimal area; blood flow velocity through tricuspid valve;

Next in stage 210 the input data set 120 is utilized as a base upon which a primary data set 126 is formed and compiled. Most preferably the primary data set 126 includes the input set of cardiac parameters 120 (obtained in stage 200), a complementary randomized data set 122, and a modeling data set 124. Optionally and preferably the primary data set 126 comprises a plurality cardiac parameters, as shown in FIG. 4A and identified in Table 1.

Most preferably the complementary data set 122 comprises randomized, system generated data set of cardiac parameters that is complementary to the input data set 120, including cardiac parameters that are not available to and/or not found in the input set 120. Most preferably the complementary data set 122 comprises parameters that are provided with randomized values within a given data range that is based on the type of parameter and its expected values and/or and within a given standard value range relative to that specific cardiac parameter. Most preferably abstractor 110 performs a check to ensure that the parameters comprising the complementary randomized data set 122 are logical. For example, internal diameter of a cardiac chamber is not larger than an external diameter of the same cardiac chamber. Optionally the validity check is provided according to a rule based and/or logical hierarchy relative to the generated parameter.

Most preferably the modeling data set 124 comprises parameters, coefficients, constants and the like mathematical data required to utilize the cardiac functions during the simulation process, for example as outlined in Table 1. Optionally and most preferably the modeling data set 124 is determined by abstractor 110 and is determined based on at least the input data set 120 and more preferably based on both the input set 120 and complementary data set 122, for example as shown in FIG. 4A.

Next in stage 220, the cardiac model abstractor 110 initiates the process for abstracting the personalized cardiac hemodynamic model based the data of the primary set 126. Most preferably the personalized cardiac model 150, as depicted in Table 4, is abstracted by evaluating the primary data set 126 with a plurality of cardiac equations 136 that most preferably, mirror the events of the cardiac cycle, therein more accurately modeling the heart forming a functional personalized cardiac hemodynamic model. Most preferably during the abstraction process the cardiac equations 136 are evaluated at a frequency of about every 10 ms.

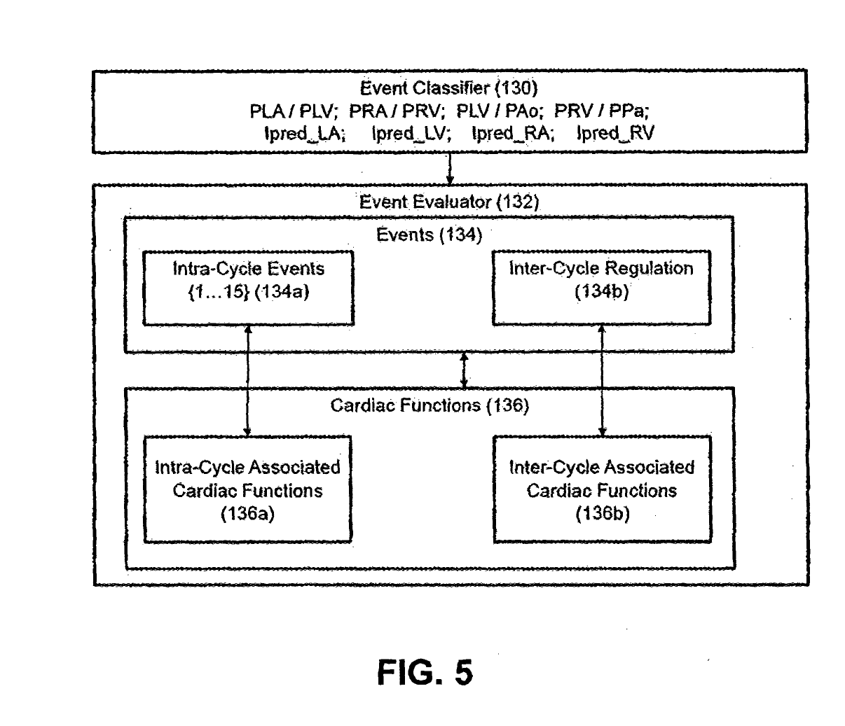

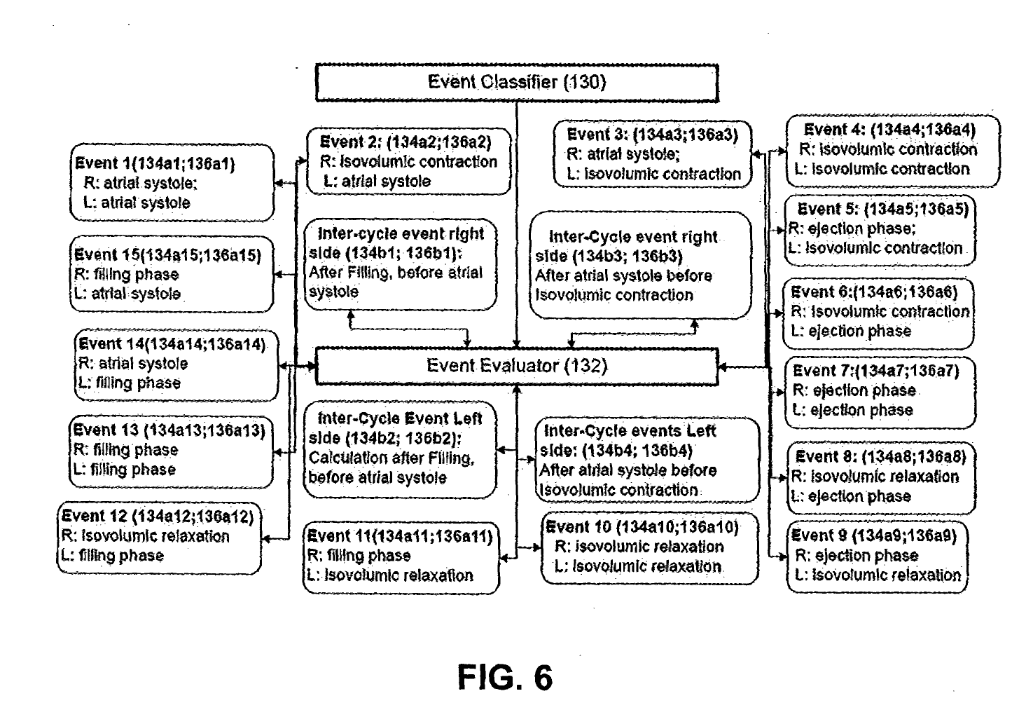

Most preferably a plurality of cardiac equations 136 are organized in such a manner so as to mirror a single cardiac cycle accounting for 15 intra cardiac cycle events 136a and a plurality of inter-cardiac cycle regulating events 136b. Most preferably an individual cardiac cycle is divided into a plurality of cardiac cycle event 134 comprising a set of 15 intra-cycle events and/or cases 134a as depicted in Table 2 and FIG. 5 and a plurality of inter-cycle regulating events 134b, also shown in FIG. 4A, FIG. 5. Most preferably each of the 15 cardiac cycle events 134a is associated with a subset of a plurality of cardiac functions 136a that are relevant to and correspond to that specific event and/or case 134a,136a FIG. 5-6. Most preferably each of the 15 cardiac cycle events 134a, individually identify an instantaneous snap shot of the cardiac cycle. The 15 cardiac cycle events collectively account for a single full cardiac cycle. Therein most preferably each of the 15 cardiac events 134a is associated with a plurality of cardiac functions 136a that describe the hearts functionality at the specific and/or instantaneous stage of the cardiac cycle.

Most preferably the 15 cases and/or events 134a comprise and account for the following events of the cardiac cycle that are defined according to the status of the right and left side respectively:

Event 1: Both sides of the heart are in atrial systole;

Event 2: Left heart still is in atrial systole, right side in isovolumic contraction;

Event 3: left side in isovolumic contraction; Right side in atrial systole;

Event 4: Both sides are in isovolumic contraction;

Event 5: Left heart in isovolumic contraction; Right side in ejection phase;

Event 6: Left side in ejection phase; Right side in isovolumic contraction;

Event 7: Both sides in ejection phase;

Event 8: Left side in ejection phase, Right side is in isovolumic relaxation;

Event 9: Left side in isovolumic relaxation, Right side in ejection phase;

Event 10: Both sides in isovolumic relaxation;

Event 11: Left side in isovolumic relaxation, Right side in filling phase;

Event 12: Left side in filling phase; Right side in isovolumic relaxation;

Event 13: Both sides in filling phase;

Event 14: Left side in filling phase, Right side in atrial systole;

Event 15: Left side in atrial systole, Right side in filling phase;

Most preferably each of the 15 cases and/or events 134a reflect the intra-cardiac cycle events 134a are associated with and evaluates a particular set of mathematical modules and/or functions 136a reiterating the specific cardiac activity.

Optionally, cardiac functions 136 provide for and are most preferably associated with hemodynamic parameters for example including but not limited to flow, circulation resistance, flow velocity, flow volume, wall elasticity, chamber volume, pressure, deformation, vessel resistance, blood density, any increments thereof, any combination thereof or the like.

Most preferably each cardiac cycle event 134 and hemodynamic parameters thereof may be associated with a plurality of cardiac function 136 selected from the group consisting of equations derived from and/or based on the following base modeling equations: elasticity equation derived from the generalized Hooke's law; passive Young moduli, active Young moduli; Euler equation, the Moens' equation, the law of conservation of mass and the law of conservation of energy.

A detailed look at the simulation stage 220, as provided in FIG. 3 describing the simulation process in sub-stages 221-225, and further schematically illustrated with reference to FIG. 4A and FIG. 5.

Simulation process preferably initiates with stage 221 where most preferably the abstractor 110 evaluates the data available in the primary data set 126 to determine which of the 15 cardiac cycle events 134 is represented by the primary data set 126. The evaluation is preferably performed by an event classifier 130, for example as shown in FIG. 4A,

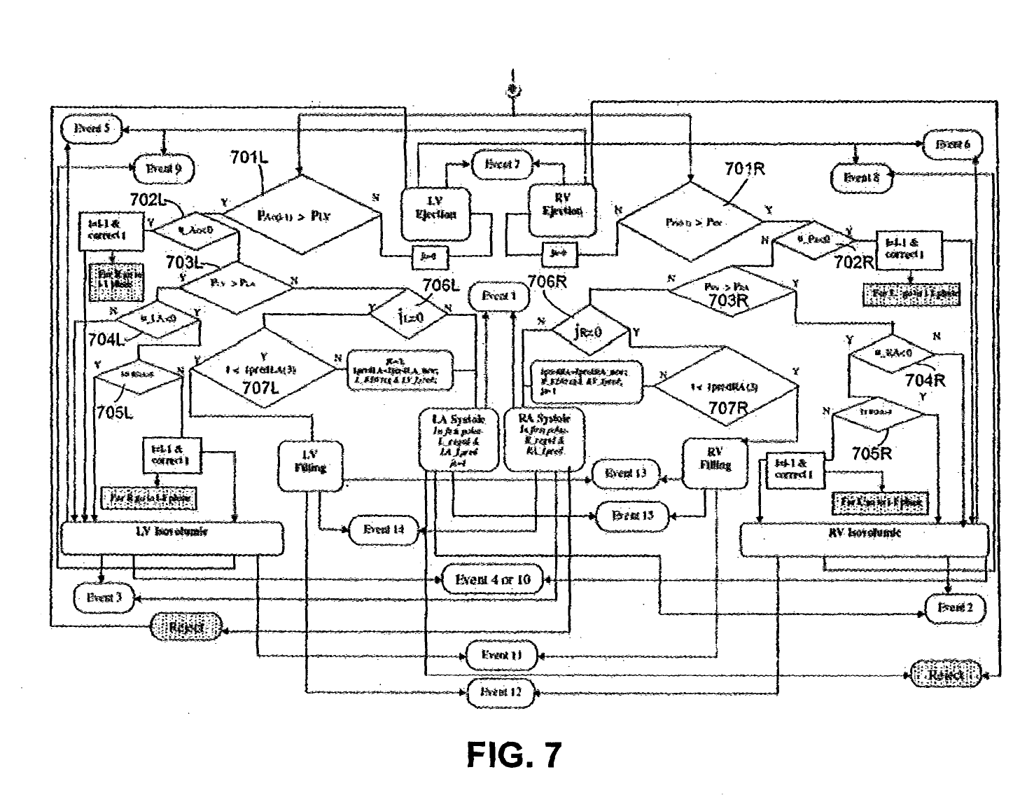

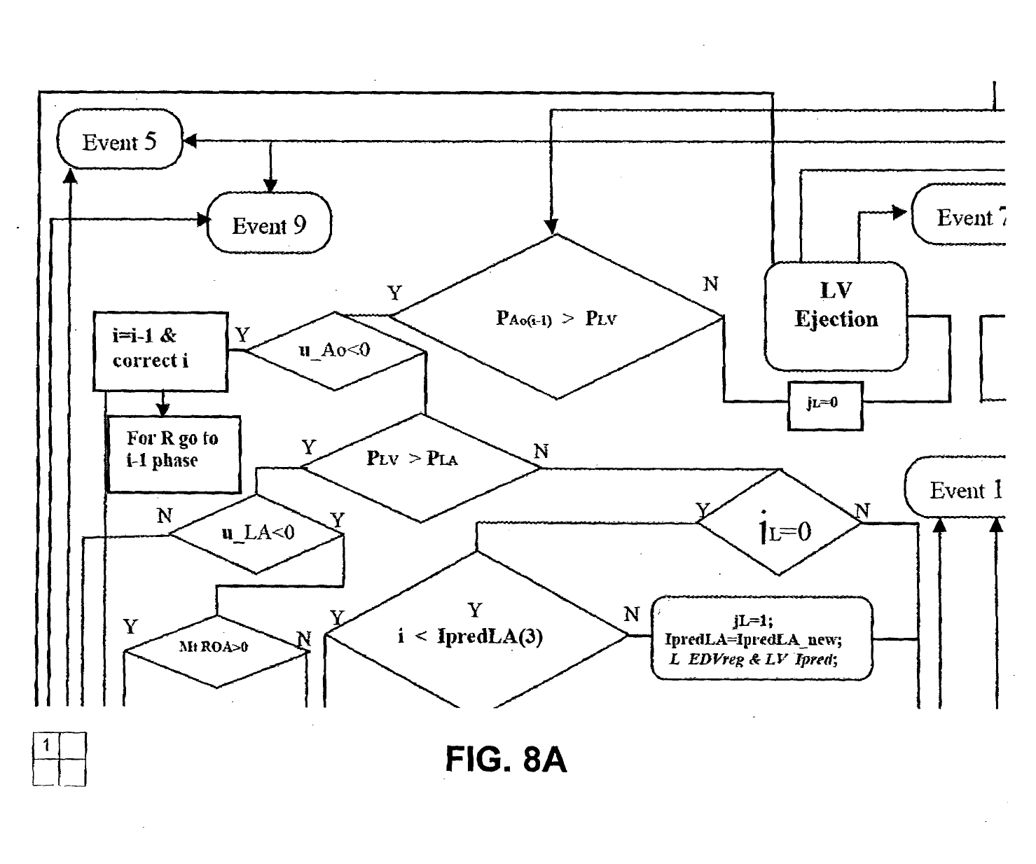

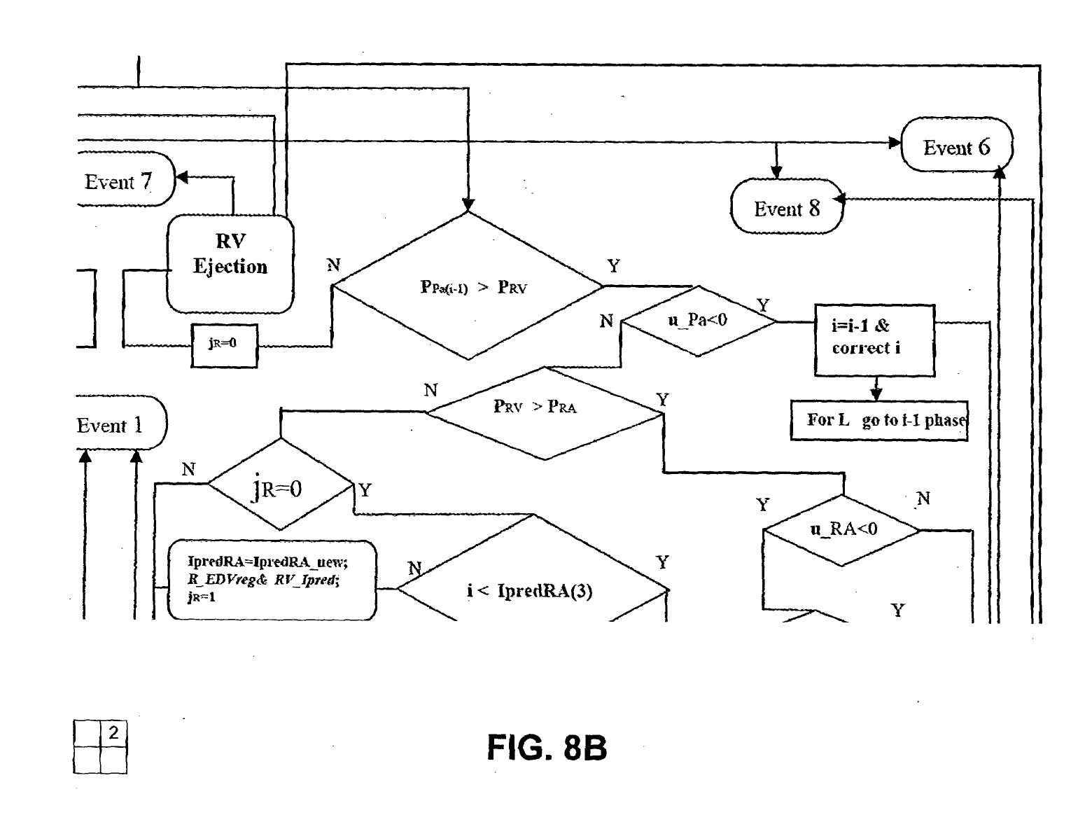

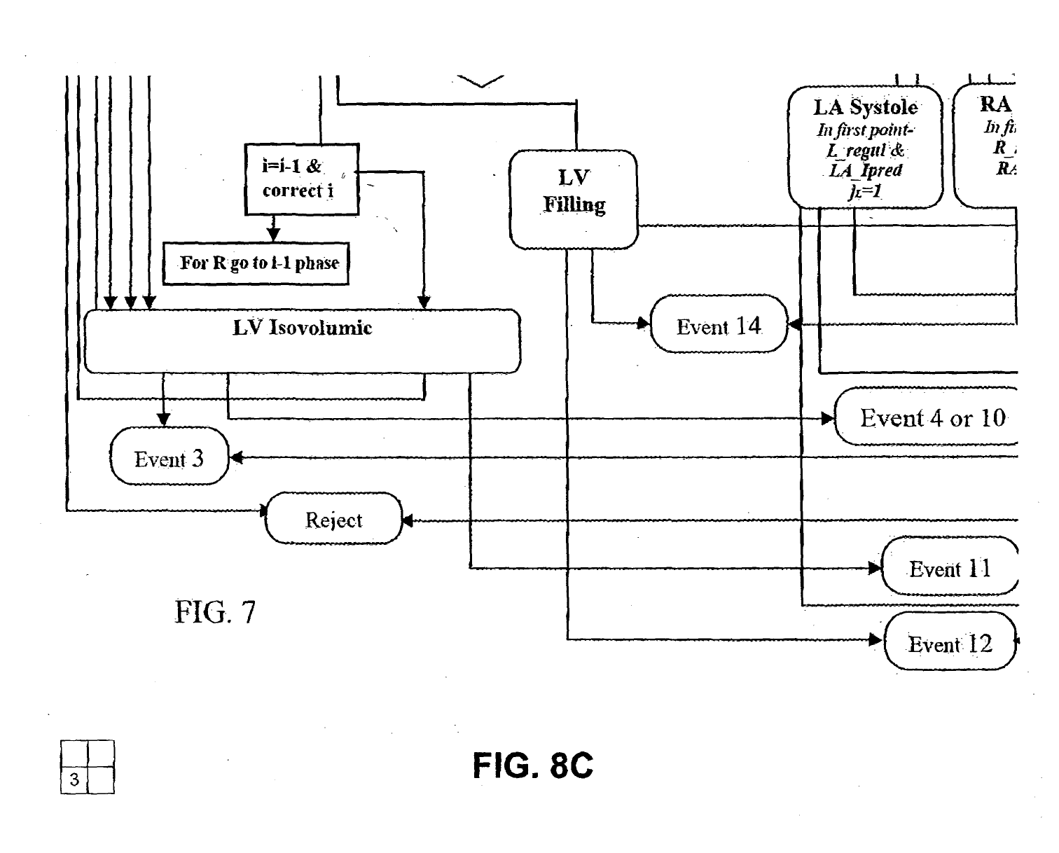

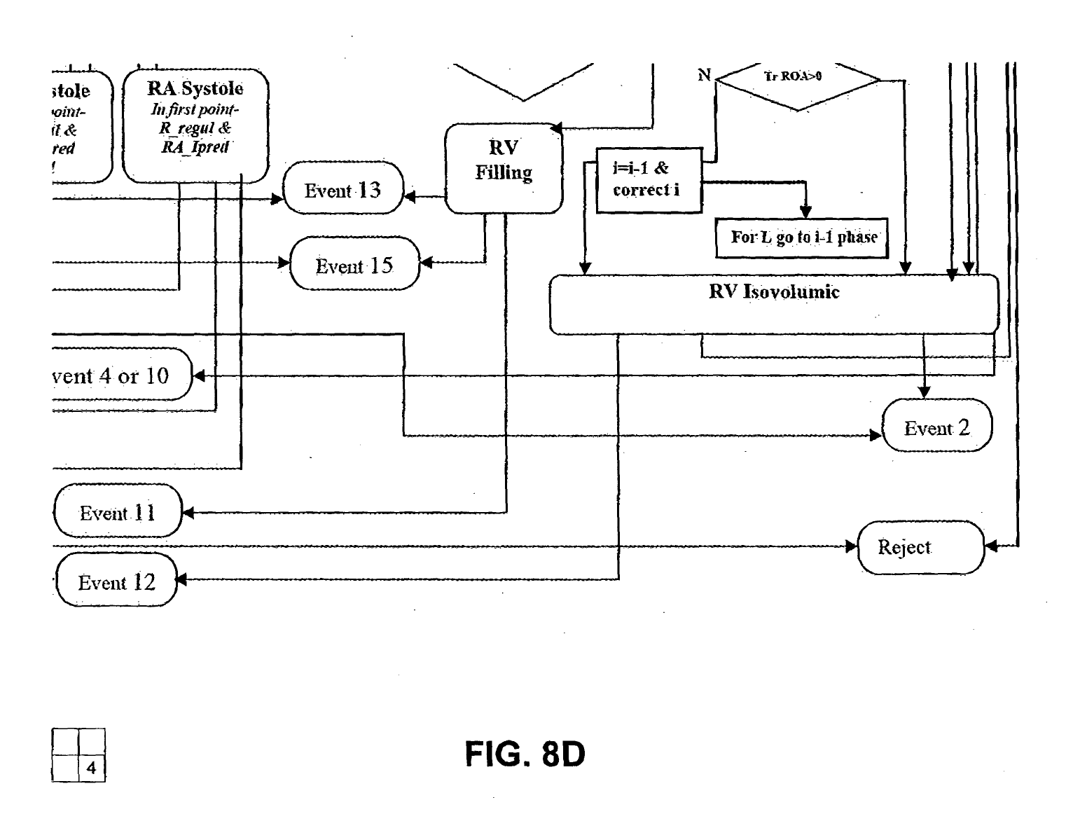

As shown in FIGS. 4A-B and FIG. 7, Event classifier 130, provides for determines the cardiac cycle event (S=Sn, n={1 . . . 15}) that may be determined by evaluating parameters forming the primary data set 126 with respect to the relative cardiac pressure in the different cardiac chambers, for example as depicted in more detail in FIG. 7. Most preferably the event classifier 130 evaluates the cardiac chamber pressure relative to one another. Therein, most preferably, event classifier 130 member of abstractor 110 determines the volume flow increments as well as the pressure ratio between cardiac chambers, for example including but not limited to PLA/PLV; PRA/PRV; PLV/PAo; PRV/PPa; Ipred_LA; Ipred_LV; Ipred_RA; Ipred_RV. Based on the relative pressure evaluation the abstractor 110 particularly classifier 130 determines which cardiac cycle event (1-15) is defined by the primary data set 126.

Next in stage 222, following cardiac cycle event determination (S=Sn, n={1 . . . 15}) abstractor 110 and in particular event evaluator 132 evaluates the respective cardiac functions associated with the given cardiac cycle event (S=Sn). For example as shown in FIG. 4A-B, FIG. 5, event evaluator 132 comprises events module 134 that correspond to cardiac functions module 136. Functions module 1336 provides for evaluating the cardiac functions specifically associated with the individual events defined in events module 134 once determined and/or classified by classifier 130.

Next in stage 223, following the evaluation of cardiac functions associated with the given cardiac cycle event (S=Sn), with evaluator 132 utilizing functions module 136 and events module 134, the parameters forming the primary parameter set 126 are updated to form an updated data set 140, which is then evaluated with evaluator module 142 for errors detection and assignment of a penalty score, Optionally model evaluator module 142 preferably evaluation data set 140 for the integrity of the individual parameters forming the data set 140, their behavior over time, temporal trends, and logical progression during the cardiac cycle.

Next in stage 224, the updated and evaluated primary data set 140 is re-evaluated with event classifier module 130 forming part of abstractor 110 to determine the next cardiac cycle event (Sₙ+1). Optionally the evaluation process may reveal that the cardiac cycle event (S=Sn) remains unchanged where (S=Sn+1=Sₙ) or that the updated data set 140 indicate that the parameters progressed to the next sequential cardiac cycle event (S=Sn+1=Sₙ₊₁, n={1 . . . 15}), or that the primary set parameters indicate that the parameters regressed to the previous sequential cardiac cycle event (S=Sn+1=Sₙ₋₁, n={1, . . . 15}). For example, the parameters (data set 140) may reflect that a current cardiac event are reflected by event 5, following the evaluation of the parameters (with evaluator 132) with the cardiac functions (136) associated with event 5 (134 and specifically 134a5 FIG. 5-6), the event may evolve to remain at the same event 5 (134a5) or change (+/−1) to an immediately following event, event 6 (134a6), or to an immediately preceding sequential event 4 (134a4).

Most preferably the reiterative evaluation process of cardiac cycle events (1-15) with events module 134 and cardiac functions module 136 and updating the data set 140 as described above, continues for at least a single full cardiac cycle, identified by cycling through all 15 events at least once, in a sequential manner from the initial stage, therein ensuring at least one full cycle. Optionally and preferably the simulation stage may provide for simulating a plurality of cardiac cycles.

Next in stage 225, the primary set has been cycled through at least one full cycle, (events 1-15), the primary set is then, evaluated with additional inter-cycle cardiac events 134b and functions 136b, FIG. 5. Most preferably the inter-cycle cardiac events and functions 134b; 136b, model pressure regulation processes. Optionally and preferably these inter-cycle cardiac events and functions, 134b;136b provided to re-evaluate and adjust the primary set as necessary for stroke volume parameters, and evaluated with respect to each of the respective 4 cardiac chambers.

Most preferably following the evaluation of the inter-cycle cardiac event and functions 134b;136b the data set 140 is updated accordingly and/or adjusted the cardiac cycle state is evaluated, with event classifier module 130, and is continuously adjusted as described in stages 222 to 224 to evaluate a plurality of cardiac functions 136 associated with the cardiac events 134 in a new cardiac cycle. Optionally a plurality of cardiac cycles may be simulated with abstractor 110.

Most preferably this reiterative evaluation, stages 222-225 continues for at least 3 and up to about 30 cardiac cycles before an initial model stability evaluation process (stage 230) is undertaken.

Next in stage 230, following at least 3 cardiac cycle simulation optionally and most preferably stable state may be evaluated with model evaluator 142, by comparing all pressure hemodynamic parameters characteristics associated with all cardiac chambers, particularly the left ventricle, right ventricle and the end diastolic pressure cardiovascular parameters, to check if they are balanced.

If the pending model has not reached a stable state the system reverts and continues simulating, stage 220, up to about to 30 cardiac cycles, until the model achieves stable state, before advancing to stage 240.

Optionally if a stable state is not reached within a 30 cycle simulation the systems reverts to the initialization, stages 210, where the primary data set is reset. Most preferably the reset primary data set is reset by forming a new complementary data set 122 and thereafter re-evaluate the modeling data set 124 forming a new primary data set 126 to abstract a new model. Optionally optimization techniques as is known in the art may be utilized to abstract an improved complementary data set 122, for example with cross entropy method.

Optionally if the primary data set 126 stable state is reached, abstractor 110 and the simulation process proceeds to evaluate the abstracted model in stage 240, with model evaluator module 142. In stage 240, the abstracted model is evaluated relative to the input data set 120 obtained in stage 200, the integrity of the individual parameters forming the primary data set and their behavior over time, temporal trends, and logical progression during the cardiac cycle.

For example, module 142 determines a penalty score that may be provided based on parametric behavior over time and/or relative to measured parameters forming the input set. For example a penalty score may be assigned relative to the pressure distribution and/or gradient about the cardiac chambers ensuring that they are logical, the volume of the chambers during the cardiac cycle; flow parameters; anatomical parameters relative to the input data set. Optionally the penalty assigned to and/or associated with a cardiac parameter may be proportional,

Most preferably the penalty is evaluated relative to a threshold. Optionally if the penalty score is above the threshold the abstraction process is reset and the systems reverts to the initialization stages, stage 210, where the primary parameter set is reset. Most preferably the reset primary data set is reset by forming a new complementary data set and thereafter a modeling data set is determined. Thereafter a new abstraction process is initialized, stages 210-240 as described hereinabove.

Optionally, if the penalty score is below the threshold the abstracted model is set, in stage 250 by setting the personalized modeling data set 150, Table 4, that may thereafter be utilized for personalized cardiac monitoring. Most preferably in stage 250 provided by personalization module 150 the abstracted model is defined, most preferably by defining the modeling parameters set 150 as system constants, most preferably such that the modeling parameters are stored in abstractor 110, that in turn determine and define the abstracted personalized cardiac model.

Most preferably stages 200 to 250 define the first phase associated with simulating and abstracting the cardiac model according to the present invention. Stages 300 to 350 define phase 2 providing the process of monitoring a plurality of cardiac parameters with the abstracted cardiac model defined in stage 250, also shown in FIG. 4B.

As shown in FIG. 4B, during and the second phase where the personalized cardiac hemodynamic model 150 abstracted in phase 1 is utilized to monitor cardiac parameters based on at least one or more measured input data set 152. Monitoring preferably initiates in stage 300 by obtaining a measured input data set 152, optionally with an optional auxiliary device 50, for example an image device, image processor, or non-imagery measuring device, or the like devices as previously described. Optionally the measured input data set 152 may be measured, either in real time monitoring with auxiliary device 50 or provided by offline monitoring, for example with stored data provided on computer readable media.

Optionally and preferably the measured input data may comprise a minimal data set 152 of cardiac parameters for example at least one or more cardiac parameters. Most preferably this may be utilized to generate a full set of cardiac parameters as an output monitoring data set 158, providing access to cardiac and hemodynamic parameters that are not readily available.

Most preferably the input measured data set 152 and the abstracted and personalized modeling data set 150 are combined to form the monitoring data set 154. Most preferably monitoring provides for elucidate cardiac parameters that are not available in the input measured data set 152, therein the monitoring data set 154 provides for extrapolating the data available in data set 152 to monitor cardiac and hemodynamic parameters that may not be readily measured or available without applying invasive measures.

Next in stage 320 monitoring is provided for by evaluating the monitoring data set 154 with the combined utility of event classifier 130, event evaluator 132 to evaluate the monitoring data set 154 with respect to cardiac cycle events 134 and their corresponding functions 136, as previously described. Most preferably during stage 320 monitoring simulation following evaluation with evaluator 132 a data updating module 138 adjusts and updates parameters forming the monitoring data set to an updated data set 156 as well as an updated data set 140 comprising updates to the parameters, coefficients, and constants utilized when evaluating cardiac equations 136. As previously described monitoring data set 154 is updated and evaluated by utilizing the cardiac functions 136 specifically associated with the 15 cardiac cycle events 134, as previously described with respect to stages 220-225 above, FIG. 3. As previously described monitoring data set 154 is preferably evaluated at a frequency of 10 ms, such that every 10 ms of data a new instance is evaluated by event classifier 130, event evaluator 132 with respect to events 134 and associated functions 136, and thereafter data set 154 is updated with data update module 138, performed for the duration of input data 152, to form the output monitoring data set 158 once the full data set 154 has been evaluated.

Next in stage 350, following the simulation provided for the full duration of the input set 152, the system outputs an output data set 158 comprising a plurality of cardiac and/or hemodynamic monitoring parameters, for example the parameters identified in Table 1 as an input or complimentary data.

Optionally and preferably the input of minimal set 152 of monitoring cardiac input parameters may for example be selected from the group consisting, of: direct pressure measurement by catheterization, Aortic lumen during cardio cycle, Ao valve opening and closing time, blood flow velocity in Aorta, blood flow velocity on Ao valve, Pulmonary Artery Lumen during cardio cycle, blood flow velocity in Pulmonary Artery, blood flow velocity on PA valve, Systolic and Diastolic Left ventricle Diameter, Mitral valve opening and closing time; Left ventricle volume during cardio cycle; Left Atrium diameters; Left Atrium Area maximal; Left Atrium area minimal; left ventricle systolic wall thickness systolic; left ventricle diastolic wall thickness; blood flow velocity through mitral valve; cardio cycle timing; Systolic right ventricle Long diameter; Diastolic right ventricle Long diameter; Systolic right ventricle short diameter; Diastolic right ventricle short diameter; Right Atrium diameter; Right Atrium maximal Area; Right atrium minimal area; blood flow velocity through tricuspid valve.

Optionally and preferably the monitoring process, from input data set 152 to monitoring output set 158, may be performed offline relative to recorded input imagery monitoring data, as previously described. Optionally monitoring may be performed online, substantially in real time during active real time monitoring of an individual, with imagery data, most preferably to provide output monitoring parameters data set 158 substantially in real time and based on a input monitoring data set 152 obtained substantially in real time.

Most preferably the cardiac parameter monitoring output 158 of stage 350 as a result of monitoring may for example include but is not limited to at least one and more preferably a plurality of output parameters selected from the group for example including but not limited to: Left Ventricle Pressure; Right Ventricle Pressure; Left Atrium Pressure; Right Atrium Pressure; Pressure in Aorta; Pressure in Pulmonary Artery; Pressure drop in the arterial, capillary and venous components of the systemic circulation; Pressure drop in the arterial, capillary and venous components of the, pulmonary circulation; Left Ventricle volume; Right Ventricle volume; Left Atrium volume; Right Atrium volume; Aortic Lumen; PA Lumen; Left ventricle Wall thickness; Right ventricle Wall thickness; Left Ventricle Intra-myocardial tensions and stresses; Right Ventricle Intra-myocardial tensions and stresses; Blood flow velocity in Aorta; Blood flow velocity in Pulmonary Artery; Blood flow passage through the Aortic valve; Blood flow passage through the PA valve; Blood flow passage through the Mitral valve; Blood flow passage through the Tricuspid valve; Systemic circulation Resistance; Pulmonary circulation Resistance; Right Ventricular pressure-volume relation; Left Ventricular pressure-volume relation; Pericardial pressure; Pericardial volume, the like or any combination thereof.

As shown in FIG. 4B, monitoring output data set 158, may undergo further evaluation and/or analysis for example with model evaluator module 160 to evaluate the quality of the output monitoring data 158.

Evaluator module 160 may provide for performing phase three according to the present invention, where the abstracted module is re-evaluated to identify any instances of cardiac remodeling that may have occurred after the personalized cardiac hemodynamic model 150 was abstracted.

Optionally phase three comprising model 150 re-evaluation may be provided following any one or more events for example including but not limited to medical intervention, change in personalized drug profile, patient profile, disease profile, physiological events, biological events, anatomical events, events that directly or indirectly affect the functionality of the cardiovascular system, the like events, or any combination thereof. For example, the model may be re-evaluated following cardiac events for example including but not limited to an infarction, stroke, seizure, heart attack, surgery, placement of a stent, angioplasty, minimally invasive surgery, valve replacement surgery, any sensed anatomical changes for example wall thickening, the like or any combination thereof.

Optionally following evaluation with module 160, output data set 158 may be communicated to output module 104. Optionally module 104 may provide for communicating output monitoring data set 158 to an optional auxiliary device 50 for example including but not limited to a display, printout, computer readable media, computer, server, smartphone, mobile communication device, healthcare system, third party device, imagery device, dedicated device, the like or any combination thereof. Optionally output module 104 may communicate output monitoring set 158 for further processing, displaying, printing, analysis or the like that may optionally be performed by an optional auxiliary device 50.

FIG. 5 shows a close up view of event classifier module 130 and event evaluator 132 that function concertedly to determine the current cardiac cycle event and thereafter to apply and evaluate the cardiac functions associated with the particular event so as to update the respective data set 126, 154, 140, 138, for example as previously described. Event classifier 130 evaluates the data set at hand to determine which event is reflected in the data. The evaluation process is depicted in the flow chart shown in FIG. 7. Classifier 130 determines the event by evaluating the relative pressure and the repolarization-depolarization timing in individual cardiac chambers on both the right and left side. Classifier 130 optionally and preferably evaluates the ratios for example including but not limited to at least one or more selected from the group consisting of: PLA/PLV; PRA/PRV; PLV/PAo; PRV/PPa; Ipred_LA; Ipred_LV; Ipred_RA; Ipred_RV, the like or any combination thereof.

Optionally and preferably classifier 130 provides for identifying both intra-cardiac cycle events (134a) or inter-cardiac cycle events (134b), therein classifier 130 may identify both intra or inter cardiac event.

Now referring to the flow chart of FIG. 7 showing an the method for depicting the different events and/or case by classifier 130. As previously described the relative pressure parameter and repolarization-depolarization timing are evaluated on both the right and left sides, as provided by PLA/PLV; PRA/PRV; PLV/PAo; PRV/PPa; Ipred_LA; Ipred_LV; Ipred_RA; Ipred_RV to identify status of each of the cardiac chambers selected from the group consisting of Atria) Systole, Isovolumic contraction, Ejection, Isovolumic relaxation and Filling. Thereafter the status of the cardiac chambers right side vs. left side, are cross reference to define the different cardiac cycle events 1 to 15, as identified in Table 2.

First in stage 701 is provided to determine the stats of the aortic valve on the left side (701L) and pulmonary artery valve on the right side (701R), respectively,

In stage 701L the evaluator determines if the Aortic Pressure (PAo) is larger than the Left ventricle pressure (PLY) to determine if the aortic valve is open or closed. If pressure is higher in the LV, the aortic valve is open indicating that the left side is characterized as being in the state of LV Ejection, which could apply to events 6, 7 or 8, pending the cardiac status on the right side, for example as outlined in Table 2. Most preferably a flag indicator jL is set to a binary value indicative of the beginning of the ejection phase, for example jL=0 as shown. Most preferably flag indicator jL is provided to accurately decipher between the correct timing and/or onset of Atrial Systole at later stages namely stage 706, as will be described. Most preferably the value of indicator jL does not change until such a time that the cardiac phase and/or status is Atrial Systole where jL=1.

If Aortic pressure is larger than left ventricle pressure, indicating that the aortic valve is closed, the method proceeds to stage 702L, described below to determine the status of the mitral valve.

In parallel stage 701R the classifier checks if the Pulmonary Artery (PPa) pressure is greater than the Right Ventricle pressure (PRV); to determine the status of the pulmonary artery valve (PAV).

If the pressure is higher in the Right Ventricle, indicating that the Pulmonary Artery valve is open, the right ventricle is characterized as being in the state of RV Ejection which could apply to events 5, 7 or 9, as outlined in Table 2, depending on the status of the left side. Most preferably a flag indicator jR is set to a binary value indicative of the beginning of the ejection phase, for example jR=0 as shown. Most preferably flag indicator jR is provided to accurately decipher between the correct timing and onset of Right Atrial Systole at later stages namely stage 706, as will be described. Most preferably the value of indicator jR does not change until such a time that the cardiac phase and/or status is Right Atrial Systole where jR=1.

If PA pressure is larger than RV pressure the indicating that the pulmonary artery valve is closed, the method proceeds to stage 702R to further decipher the status of the tricuspid valve,

Next in stage 702R/L the classifier 130 respectively determines if the maximum blood velocity through the aorta on the left side and the pulmonary artery on the right is below or equal to zero. If the velocity through the respective valve is below or equal to zero the cardiac status of the right side is isovolumic relaxation corresponding to events 8, 10, 12 while the left side status is also in isovolumic relaxation corresponding to events 9, 10, 11, as outlined in Table 2.

However if maximum blood flow through the respective valves is positive, the method proceed to stage 703 to further decipher the cardiac status.

Next in stage 703, the pressure in the ventricles is compared to the pressure in the atrium on the respective sides 703R, 703L to evaluate if the pressure in the ventricle is larger than that in the atrium. This evaluation provides for inferring the status of the mitral valve (left side) and tricuspid valve (right side) to determine if the valve is open or closed.

If pressure is higher in the Atrium, mitral valve is open, the cardiac chamber status is either Ventricle Filling or Atrial Systole, this will be determined following evaluation of stage 706, discussed below.

If pressure is higher in the Ventricle then the state is determined to be in Isovolumic where the exact status of isovolumic relaxation or contraction is determined in stage 704.

Next in stage 704 flow velocity through the atrium (mitral valve or tricuspid valve) is respectively evaluated on both right and left sides. If flow is positive (above zero) the status is determined to be isovolumic relaxation corresponding to cases 8, 10, 12 on the right and events 9, 10, 11 on the left.

If the Atrial flow velocity is determined to be negative, and/or equal to zero the status is determined to be isovolumic contraction corresponding to events 2, 4, 6 on the right and events 3, 4, 5 on the left.

Next stage 705 provides for identifying any instances of regurgitation through the respective mitral or tricuspid valve, as the cardiac status is isovolumic contraction.

Next in stage 706 following stage 703, where as described above, the classifier determined that the pressure is higher in the Atrium than the ventricle, therein the mitral valve on the Left side is open, while the tricuspid valve on the Right side is open, and therefore the cardiac chamber status is either Ventricle Fining or Atrial Systole. In order to decipher between ventricle filling and atrial systole we utilized the indicator jR/jL.

First in stage 706 the indicator jL and jR are respectively checked, to identify the atrial systole status. If jL/jR is indicative of atrial systole then the status is associated with events 1, 3, 14 on the right side and events 1, 2 and 15 on the left.

If indicator jL/jR does not indicate the atrial systole, where jL/jR=0 then stage 707 is utilized to determine if cardiac status is in systole or filling, as shown.

In stage 707 classifier 130 determines the repolarization-depolarization timing of the atrium, Ipred_RA and Ipred_LA is evaluated to determine if the current time point is before or after depolarization.

If the current time point is before depolarization then the status is determined to be ventricular filling, corresponding to events 11, 13, 15 on the right side and events 12, 13, 14 on the left, as shown in Table 2.

If the current time point is equal to or after depolarization then the status is determined to be atrial systole corresponding to events 1, 3, 14 on the right side and events 1, 2, 15 on the left, as shown in Table 2. At this time the indicator jL/jR are updated to indicate to the system the status of atrial systole, providing it a value of jL/jR=1.

Referring back to FIG. 5, following the event determination with event classifier module 130, event evaluator 132 provides an iterative process that interfaces and correlates between events module 134 and cardiac function module 136. Event module 134 provides for identifying and mapping and/or correlating the event to a subset of a plurality of cardiac functions in module 136 that are specific to the particular event. Events sub-module 134 identifies the event type as depicted by classifier 130 and checks if the data set requires inter-cycle regulation processing with sub-module 134b or if to apply intra-cycle processing with module 134a. Module 134 determines the required sub-module 134a, 134b depending on the event timing relative to a full cycle, that is if a full cardiac cycle has been processed, for example at least one round through events 1-15, then sub-module 134b is activated; while if the event is shown to be within a cycle, for example events have not cycled through all event 1-15, then sub-module 134a is utilized.

Cardiac functions module 136 comprises a library of plurality of cardiac functions that model cardiac hemodynamic activity for example including but not limited to elasticity equation derived from the generalized Hooke's law; passive Young moduli, active Young moduli; Euler equation, the Moens' equation, the law of conservation of mass and the law of conservation of energy, any derivations or combinations thereof.

Cardiac functions module 136 functions in conjunction with events module 134 to evaluate and update the data set through individual events. Accordingly cardiac functions module 136 comprises sub-module 136a to evaluate intra-cardiac cycle event and sub-module 136b to evaluate inter-cardiac cycle events by applying the appropriate set of cardiac functions associated with the particular event, for example as depicted in Table 3.

Sub module 136b may be activated after a full cycle has been rendered and most preferably when events module 134 identifies instances where the data set reflects the cardiac status as being in either of the following states: after filling and before atrial systole and/or after atrial systole before isovolumic contraction on either of the right side or left side. Most preferably sub module 136b comprises inter-cycle cardiac functions for each event and for each side, may for example provide for determining the Ipred_RV, Ipred_LV, Ipred_RA, Ipred_LA, R_EVDreg (right side pre-systolic volume-related regulation), L_EVDreg (left side pre-systolic volume-related regulation), R_regul (right side pressure-related regulation), L_regul (left side pressure-related regulation).

Following evaluation of the data set with the cardiac functions in module 136 selected based on the events module 134, evaluator 132 updates and communicates the parameters of the data set, according to the results of the cardiac functions.

FIG. 6 provides a further depictions of the coordinated functions of the event classifier 130 and event evaluator 132 controlled with the abstractor 110 of the present invention. FIG. 6 shows the type of intra-cycle events 1 . . . 15 associated with their particular event sub-module 134a1-15 relative to each event and the corresponding cardiac functions disposed in sub-module 136a1-15. Similarly the inter-cycle events of both the right and left side are depicted relative to their respective events sub-module 134b1-4 and corresponding cardiac functions 136b1-4.

While the invention has been described with respect to a limited, number of embodiments, it will be appreciated that many variations, modifications and other applications of the invention may be made.

What is claimed is:

1. A method for abstracting a personalized cardiac hemodynamic model of the heart, the method comprising:
a. Obtaining an input data set of a plurality of measured cardiac parameters;
b. Generating a complementary randomized data set to complement said input data set, and a modeling data set;
c. Formulating a primary data set including said input data set, said complementary data set and said modeling data set;
d. simulating said primary data set with a cardiac model abstractor to abstract a personalized cardiac hemodynamic model; said cardiac model abstractor is characterized in that said primary data set is evaluated and adjusted by simulating a plurality of cardiac cycles to obtain said personalized cardiac model; wherein each cardiac cycle is divided into 15 cardiac cycle events, each event mirroring a snap shot of the cardiac chamber's status during a cardiac cycle; and wherein each cardiac cycle event is represented by, and associated with, a plurality of cardiac functions that model said individual cardiac cycle event;
e. wherein said primary data is sequentially evaluated through said plurality of said cardiac cycle events with said plurality of cardiac functions such that after each cardiac cycle event said primary data set is updated and adjusted forming an updated data set;
f. performing said simulation through a plurality of cardiac cycles until a stable state criteria is reached; and
g. evaluating said updated data set in light of said input data set relative to an error threshold.

2. The method of claim 1 further comprising, evaluating said primary data set with a plurality of inter-cycle cardiac functions, between two sequential cardiac cycles, wherein said inter-cycle cardiac functions are regulating cardiac functions.

3. The method of claim 2 wherein said inter-cycle cardiac functions are associated with inter-cycle events and evaluate when the status of the cardiac chambers on the left or right side of the heart is: after filling and before atrial systole or after atrial systole before isovolumic contraction.

4. The method of claim 1 wherein said 15 intra-cardiac cycle events are selected from the group consisting of both hearts are in atrial systole; left heart is in atrial systole, the right heart is in isovolumic contraction; the right heart is in atrial systole, the left heart is on isovolumic contraction; both hearts are in isovolumic contraction; the left heart is in isovolumic contraction, the right heart is in ejection phase; the right is in isovolumic contraction, the left heart is in ejection phase; both hearts are in ejection phases; the left heart is in ejection phase, the right heart is in isovolumic relaxation; the right heart is in ejection phase, the left heart is in isovolumic relaxation; both hearts are in isovolumic relaxation; the left heart is in isovolumic relaxation, the right heart is in filling phase; the right heart is in isovolumic relaxation, the left heart is in filling phase; both hearts are in filling phases; the left heart is in filling phase, the right heart is in atrial systole; the right heart is in filling phase, the left heart is in atrial systole.

5. The method of claim 1 wherein each cardiac cycle event is associated with a plurality of cardiac functions reflecting the specific cardiac cycle event and reiterating the specific cardiac activity.

6. The method of claim 1 wherein said input data set is obtained by way of image processing of at least one or more imagery signals selected from the group consisting of: ultrasound, Doppler ultrasound, echocardiogram, angiogram, CT, MRI, PET, the like or any combination thereof.

7. The method of claim 1 wherein said input data set comprise measurements obtained with at least one or more devices selected from the group consisting of: sphygmomanometer, blood pressure device, catheterization, implanted device, electrocardiograph (‘ECG’ or ‘EKG’), laboratory testing, blood works, ultrasound, Doppler ultrasound, echocardiogram, angiogram, CT, MRI, PET, or any combination thereof.

8. The method of claim 1 wherein said input data set comprises at least one or more selected echocardiogram parameters selected from the group consisting of: aortic lumen during cardio cycle, Ao valve opening and closing time, blood flow velocity in aorta, blood flow velocity on Ao valve, pulmonary artery lumen during cardio cycle, blood flow velocity in pulmonary artery, blood flow velocity on pa valve, systolic and diastolic left ventricle diameter, mitral valve opening and closing time; left ventricle volume during cardio cycle; left atrium diameters; left atrium area maximal; left atrium area minimal; left ventricle systolic wall thickness systolic; left ventricle diastolic wall thickness; blood flow velocity through mitral valve; cardio cycle timing; systolic right ventricle long diameter; diastolic right ventricle long diameter; systolic right ventricle short diameter; diastolic right ventricle short diameter; right atrium diameter; right atrium maximal area; right atrium minimal area; blood flow velocity through tricuspid valve; any combination thereof.

9. A method for monitoring cardiac parameters with the personalized cardiac hemodynamic model abstracted according to claim 1, wherein at least one and up to seven monitoring input cardiac parameters are simulated with said personalized cardiac model to produce a set of monitored output cardiac parameters.

10. The method of claim 8 wherein said set of output cardiac parameters are selected from the group consisting of: left ventricle pressure; right ventricle pressure; left atrium pressure; right atrium pressure; pressure in aorta; pressure in pulmonary artery; pressure drop in the systemic circulation; pressure drop in the arterial systemic circulation; pressure drop in the capillary systemic circulation; pressure drop in the venous components of the systemic circulation; pressure drop in the pulmonary circulation; pressure drop in the arterial pulmonary circulation; pressure drop in the capillary pulmonary circulation; pressure drop in the venous components of the pulmonary circulation; left ventricle volume; right ventricle volume; left atrium volume; right atrium volume; aortic lumen; pa lumen; left ventricle wall thickness; right ventricle wall thickness; left ventricle intra-myocardial tensions and stresses; right, ventricle intra-myocardial tensions and stresses; blood flow velocity in aorta; blood flow velocity in pulmonary artery; blood flow passage through the aortic valve; blood flow passage through the pa valve; blood flow passage through the mitral valve; blood flow passage through the tricuspid valve; systemic circulation resistance; pulmonary circulation resistance; right ventricular pressure-volume relation; left ventricular pressure-volume relation; pericardial pressure; pericardial volume, any combination thereof.

11. The method of claim 1 wherein said simulation is initialized by determining the initial cardiac cycle stage by evaluating said primary data set to determine the volume flow increments and pressure ratios between cardiac chambers.

12. The method of claim 11 wherein said volume flow increments and pressure ratios between cardiac chambers is provided by the cardiac equations selected from the group consisting of: PLA/PLV; PRA/PRV; PLV/PAo; PRV/PPa; Ipred_LA; Ipred_LV; Ipred_RA; Ipred_RV.

13. The method of claim 1 wherein said personalized cardiac hemodynamic model is represented by said modeling data set.

14. The method of claim 1 wherein said plurality of simulated cardiac cycles is at least 3 and up to about 30 cycles.

15. A system for abstracting a personalized cardiac hemodynamic model of a user's heart, the system comprising an input module, a cardiac hemodynamic model abstractor and an output module, the system characterized in that said abstractor abstracts a personalized cardiac model based on primary data set comprising a plurality of cardiac parameters, wherein at least a portion of said cardiac parameters are provided by said input module; said primary data set is processed with said abstractor by utilizing an event classifier module provided to identify a cardiac cycle event represented by said primary data, wherein said cardiac cycle events are selected from a group of at least 15 intra-cycle events wherein each event mirrors a snap shot of the cardiac chamber's status during a cardiac cycle, and wherein each cardiac cycle event is associated with, a plurality of cardiac functions that model said individual cardiac cycle event; said cardiac cycle events and said associated cardiac functions provide for evaluating the parameters of said primary data set with an event evaluator module to abstract said personalized cardiac hemodynamic model; and a model evaluating module for evaluating said abstracted personalized cardiac hemodynamic model.

16. The system of claim 15 wherein said event classifier further classifies inter-cycle cardiac regulating events occurring between two sequential cardiac cycles.

17. The system of claim 15 wherein said event classifier module and said event evaluator modules provide for inferring a plurality of cardiac parameters from an input data set comprising at least one cardiac parameter and said personalized cardiac hemodynamic model providing a monitoring output data set.

18. The system of claim 17 wherein said inferred plurality of cardiac parameters are processed or communicated to an auxiliary device with said output module.

19. The system of claim 15 wherein said input module comprises an image processor for processing cardiac imagery data to produce a plurality of cardiac parameters, wherein said cardiac imagery data is selected from at least one or more of the group consisting of ultrasound, Doppler ultrasound, echocardiogram, angiogram, CT, MRI, PET, the like or any combination thereof.

20. The system of claim 17 wherein said monitoring output data set comprises an output set of cardiac parameters selected from the group consisting of: left ventricle pressure; right ventricle pressure; left atrium pressure; right atrium pressure; pressure in aorta; pressure in pulmonary artery; pressure drop in the systemic circulation; pressure drop in the arterial systemic circulation; pressure drop in the capillary systemic circulation; pressure drop in the venous components of the systemic circulation; pressure drop in the pulmonary circulation; pressure drop in the arterial pulmonary circulation; pressure drop in the capillary pulmonary circulation; pressure drop in the venous components of the pulmonary circulation; left ventricle volume; right ventricle volume; left atrium volume; right atrium volume; aortic lumen; pa lumen; left ventricle wall thickness; right ventricle wall thickness; left ventricle intra-myocardial tensions and stresses; right ventricle intra-myocardial tensions and stresses; blood flow velocity in aorta; blood flow velocity in pulmonary artery; blood flow passage through the aortic valve; blood flow passage through the pa valve; blood flow passage through the mitral valve; blood flow passage through the tricuspid valve; systemic circulation resistance; pulmonary circulation resistance; right ventricular pressure-volume relation; left ventricular pressure-volume relation; pericardial pressure; pericardial volume, any combination thereof.

21. The system of claim 18 wherein said output is communicated to a processing center or an auxiliary device.

22. The system of claim 22 wherein said auxiliary device is selected from the group consisting of computer, mobile communication device, server, ultrasound system, electrocardiogram, catheterization, imaginary data, imagery device, MRI, CT, PET.

23. A machine-readable medium including instructions for abstracting a personalized cardiac hemodynamic model by performing the method of claim 1.

24. A method executed by a programmable computer to abstract a personalized cardiac hemodynamic model by performing the method of claim 1.
